(19) United States
(12) Patent Application Publication
(10) Pub. No.: US 2004/0197408 A1
(43) Pub. Date: Oct. 7, 2004

(54) Amino acids in micelle preparation

(75) Inventor: David M. Gravett, Vancouver (CA)

Correspondence Address: SEED INTELLECTUAL PROPERTY LAW GROUP PLLC, 701 FIFTH AVENYUE, SUITE 6300, SEATTLE, WA, 98104-7092, US

(73) Assignee: Angiotech International AG, Zug (CH)

(21) Appl. No.: 10/748,700

(22) Filed: Dec. 29, 2003

Related U.S. Application Data
(60) Provisional application No. 60/437,385, filed on Dec. 30, 2002.

(51) Int. Cl. A61K 9/14

(52) U.S. Cl. 424/486

(57) ABSTRACT

Formulations are formed of a diblock copolymer (X—Y) having a hydrophilic block X comprising residues of monomer x, and a hydrophobic block Y comprising residues of monomer y; an additive selected from an amino acid and an oligopeptide. Upon admixture with water, the formulations form drug delivery vehicles, preferably in micellar form. The inclusion of amino acid and/or oligopeptide provides for the formation of micelles at an enhanced rate, and/or provides the formation of micelles having an enhanced ability to incorporate drug(s), and/or provides the formation of micelles having advantageous physical characteristics.

### CROSS REFERENCE TO RELATED APPLICATIONS

This application claims the benefit of U.S. Provisional Patent Application No. 60/437,385, filed Dec. 30, 2002, where this provisional application is incorporated herein by reference in its entirety.

### TECHNICAL FIELD

The present invention relates to drug delivery vehicles, more particularly to micellar drug delivery vehicles, to precursor compositions for drug delivery vehicles, and to methods of making and using such vehicles and precursors.

### BACKGROUND OF THE INVENTION

Many pharmaceutically active compounds, i.e., drugs, intended for administration to a mammal, have limited solubility in water. This hydrophobic property often makes it difficult to formulate a drug so that it exhibits a satisfactory bioavailability profile in vivo. Poor bioavailability may lead to ineffective therapy, the need for higher dosing and/or undesirable side effects.

Delivery vehicles for hydrophobic pharmaceutically active compounds have been described. See, e.g., U.S. Pat. Nos. 6,096,338; 6,077,543; 5,843,891; 5,834,019; 5,827,541; 5,776,486; 5,645,856; 5,478,860; and 5,430,021. Micellar drug delivery systems have been used to deliver a hydrophobic drug to a subject (see, for example, Zhang, X. et al. International Journal of Pharmaceutics 132:195-206 (1996)).

There exists a need in the art for improved vehicles for the delivery of hydrophobic drugs, and for methods of forming improved vehicles.

### SUMMARY OF THE INVENTION

The present invention provides improved, drug-containing compositions that may be combined with an aqueous medium to form a macroscopically homogeneous, fluid mixture wherein the drug is dispersed throughout the mixture, typically within micelles. The compositions of the present invention are particularly advantageous in that they may form micelles at an enhanced rate, have an enhanced ability to incorporate drug(s); and/or have advantageous physical characteristics that render the compositions particularly easy to make and/or handle. Water-soluble polymeric drug delivery systems often require some period of time to constitute the solid materials into the appropriate solution that can be delivered to a animal or human. The present invention addresses this shortcoming with existing systems. In addition, the present invention provides precursors to these compositions, methods to make the precursors and/or compositions, and other related compositions and methods as described below.

In one aspect, the present invention provides a composition that includes:

(a) a micelle-forming biocompatible diblock copolymer (X—Y) having a hydrophilic block X comprising residues of monomer x, and a hydrophobic block Y comprising residues of monomer y;

(b) amino acid; and

(c) a hydrophobic drug;

with the proviso that the composition forms a micellar solution in water.

Preferably, the composition does not form a hydrogel upon combination of the composition with aqueous media.

In another aspect, the present invention provides a composition that includes:

(a) a micelle-forming biocompatible diblock copolymer (X—Y) having a hydrophilic block X comprising residues of monomer x, and a hydrophobic block Y comprising residues of monomer y;

(b) oligopeptide; and

(c) a hydrophobic drug;

with the proviso that the composition forms a micellar solution in water.

In another aspect, the present invention provides a composition that includes:

(a) a biocompatible diblock copolymer (X—Y) having a block X comprising residues of monomer x, and a block Y comprising residues of monomer y, the block X being more hydrophilic than the block Y;

(b) an additive selected from amino acid and oligopeptide; and

(c) a hydrophobic drug;

with the proviso that the composition forms a micellar solution in aqueous media.

In another aspect, the present invention provides a composition that includes:

(a) a micelle-forming biocompatible block copolymer having a Y—X—Y or X—Y—X structure, wherein the copolymer has a hydrophilic block X comprising residues of monomer x, and a hydrophobic block Y comprising residues of monomer y;

(b) amino acid; and

(c) a hydrophobic drug;

with the proviso that the composition forms a micellar solution in water.

In another aspect, the present invention provides a composition that includes:

(a) a micelle-forming biocompatible block copolymer having a Y—X—Y or X—Y—X structure, wherein the copolymer has a hydrophilic block X comprising residues of monomer x, and a hydrophobic block Y comprising residues of monomer y;

(b) oligopeptide; and

(c) a hydrophobic drug;

with the proviso that the composition forms a micellar solution in water.

In another aspect, the present invention provides a composition that includes:

(a) a biocompatible block copolymer having a Y—X—Y or X—Y—X structure, wherein the copolymer has a block X comprising residues of monomer x, and a block Y comprising residues of monomer y, the block X being more hydrophilic than the block Y;

(b) an additive selected from amino acid and oligopeptide; and

(c) a hydrophobic drug;

with the proviso that the composition forms a micellar solution in aqueous media.

In one embodiment, the block X includes residues of one or more monomers selected from (meth)acrylic acid, styrene sulfonate, 2-acrylamido-2-methyl propane sulfonic acid, acrylamide, vinylpyrrolidone, saccharide, and amino acid. In another embodiment, the block X includes residues of alkylene oxide. In certain embodiments, the block X includes poly(alkylene oxide), such as, poly(ethylene oxide) or terminal C₁-C₆ alkyl ethers of poly(ethylene oxide), e.g., methoxy polyethylene oxide.

The block Y may include residues of monomers selected from methacrylic acid, esters of methacrylic acid, esters of acrylic acid, and vinyl acetate. In some embodiments, the block Y includes residues of monomers selected from lactic acid and reactive equivalents thereof, glycolic acid and reactive equivalents thereof, caprylic acid and reactive equivalents thereof, trimethylene carbonate, 1,4-dioxane-2-one, and 1,5-dioxepan-2one. In certain embodiments, the block Y is poly-DL-lactide-co-glycolide or poly-DL-lactide.

In one aspect, the block X includes residues of monomers selected from alkylene oxide, acrylic acid, vinyl pyrrolidone, saccharide, and amino acid, and block Y comprises residues of monomers selected from lactide or reactive equivalents thereof, glycolide or reactive equivalents thereof, caprolactone or reactive equivalents thereof, hydrophobic amino acid, carbonate, and vinyl acetate. In another aspect, block X includes residues of alkylene oxide, and block Y includes residues of monomers selected from lactide or reactive equivalents thereof, glycolide or reactive equivalents thereof, caprolactone or reactive equivalents thereof, trimethylene carbonate, 1,4-dioxane-2-one, and 1,5-dioxepan-2one. In another aspect, block X includes residues of alkylene oxide and block Y includes residues of monomers selected from lactide or reactive equivalents thereof and glycolide or reactive equivalents of glycolide. In yet another aspect, block X includes residues of ethylene oxide, and block Y includes residues of lactide. In yet another aspect, block X is methoxy polyethylene oxide and block Y is poly(DL-lactide).

Compositions having 100 parts of diblock copolymer may include 40-90 parts hydrophilic polymer X and 60-10 parts hydrophobic polymer Y, or 40-80 parts hydrophilic polymer X and 60-20 parts hydrophobic polymer Y, or 50-70 parts hydrophilic polymer X and 50-30 parts hydrophobic polymer Y, or about 60 parts hydrophilic polymer X and about 40 parts hydrophobic polymer Y.

The diblock copolymer may have a number average molecular weight of about 1,000 to about 10,000 g/mol, or about 2,000 to about 5,000 g/mol, or about 2,500 to about 3,500 g/mol.

In one aspect, the composition may include an amino acid or an oligopeptide having a water-solubility of greater than about 2.5 g per 100 g water at 25° C. Examples of amino acids having such a water solubility include, e.g., the L and D isomers of alanine, arginine, asparagines, cysteine, glutamine, glycine, histidine, isoleucine, lysine, methionine, phenylalanine, proline, threonine, and valine.

The instant compositions may include a naturally occurring amino acid (e.g., a L or D isomer of alanine, arginine, asparagine, aspartic acid, cysteine, glutamine, glutamic acid, glycine, histidine, isoleucine, leucine, lysine, methionine, phenylalanine, proline, serine, threonine, tryptophane, tyrosine, or valine) or a non-naturally occurring amino acid (e.g., β-alanine, α-amino butyric acid, γ-amino butyric acid, γ-(aminophenyl) butyric acid, α-amino isobutyric acid, ε-amino caproic acid, 7-amino heptanoic acid, β-aspartic acid, aminobenzoic acid, aminophenyl acetic acid, aminophenyl butyric acid, γ-glutamic acid, cysteine (ACM), ε-lysine, ε-lysine, (A Fmoc), methionine sulfone, norleucine, norvaline, omithine, d-ornithine, p-nitro-phenylalanine, hydroxy proline, 1,2,3,4,-tetrahydroisoquinoline-3-carboxylic acid, or thioproline).

The composition may further include MePEG. Optionally, in various aspects, the MePEG may have a molecular weight of 200-750 g/mol, or 550-2000 g/mol, or 750-5000 g/mol, or 200-5000 g/mol.

The composition may include about 1 to about 5 parts block copolymer per each 1 part additive, on a weight basis.

The hydrophobic drug may be, e.g., a chemotherapeutic, antibiotic, antimicrobial, antimicrotubule, anti-inflammatory, immunosuppressant, or antiproliferative drug. In one aspect, the drug may be paclitaxel or a paclitaxel derivative or analogue.

The instant composition may further include a buffering constituent, such as a phosphate salt. In one aspect, the composition may include 10-90 parts diblock copolymer, 10-70 parts additive selected from amino acid and oligopeptide, 1-15 parts paclitaxel and 1-20 parts phosphate salt. In another aspect, the composition may include about 50-80 parts of diblock copolymer, about 10-40 parts additive selected from amino acid and oligopeptide, about 8 parts paclitaxel and about 18 parts phosphate salt, the parts in weight totaling 100. In yet another aspect, the composition may include about 55-75 parts diblock copolymer, about 15-35 parts additive selected from amino acid and oligopeptide, about 7 parts paclitaxel and about 11 parts phosphate salt, the parts in weight totaling 100.

In optional aspects, the composition has little or no water. For example, the composition may have less than 5% moisture content or less than 0.5% moisture content. Optionally, a composition of the invention may be produced through lyophilization of a micellar solution. The composition may optionally include a bacteriacidal or bacteriostatic compound, or an antioxidant or a coloring agent, or a combination of two or more of these components.

Optionally, a composition of the present invention may be sterile. In one aspect, the present invention provides a composition, as briefly stated above, that is packaged within a container (e.g., a glass or plastic container with a sealed closure) that maintains the sterility of the composition for a sufficient time to be useful, e.g., one week. The present compositions may optionally be treated according to a sterilization process, such as a process selected from sterile filtration, sterilization with ethylene oxide, and sterilization with ionizing radiation. The packaging may further include a sufficient volume of empty space to allow for the addition of water in a sufficient amount to produce a micelle-containing composition. Optionally, the packaging is substantially opaque to UV or visible light and may additionally be substantially impervious to oxygen from air.

The composition may further include water to form an aqueous composition, the aqueous composition comprising micelles. In one aspect, the composition may include:

(a) a biocompatible diblock copolymer (X—Y) having a hydrophilic block X and a hydrophobic block Y;

(b) amino acid,

(c) a hydrophobic drug; and

(d) water;

wherein the composition comprises micelles.

In another aspect, the composition may include:

(a) a biocompatible diblock copolymer (X—Y) having a hydrophilic block X, and a hydrophobic block Y;

(b) an oligopeptide;

(c) a hydrophobic drug; and

(d) water;

wherein the composition comprises micelles.

In yet another aspect, the composition may include:

(a) a biocompatible diblock copolymer (X—Y) having a hydrophilic block X, and a hydrophobic block Y;

(b) two different amino acids;

(c) a hydrophobic drug; and

(d) water;

wherein the composition comprises micelles.

In yet another aspect, the composition may include:

(a) a biocompatible block copolymer having a X—Y—X or Y—X—Y structure, wherein the copolymer has a hydrophilic block X and a hydrophobic block Y;

(b) amino acid,

(c) a hydrophobic drug; and

(d) water;

wherein the composition comprises micelles.

In yet another aspect, the composition includes:

(a) a biocompatible block copolymer having a X—Y—X or Y—X—Y structure, wherein the copolymer has a hydrophilic block X, and a hydrophobic block Y;

(b) an oligopeptide;

(c) a hydrophobic drug; and

(d) water;

wherein the composition comprises micelles.

In yet another aspect, the composition may include:

(a) a biocompatible block copolymer having a X—Y—X or Y—X—Y structure, wherein the copolymer has a hydrophilic block X, and a hydrophobic block Y;

(b) two different amino acids;

(c) a hydrophobic drug; and

(d) water;

wherein the composition comprises micelles.

In one aspect, the present invention provides a method for forming a drug delivery vehicle. The method includes sequentially providing a non-aqueous composition comprising diblock copolymer, additive selected from amino acid and oligopeptide, and hydrophobic drug as described herein; and adding aqueous media (e.g., water or buffer) to the composition to form a micelle-containing composition.

In another aspect, a method is provided for forming a composition in accordance with the invention that includes combining a hydrophobic drug, an additive selected from amino acid and oligopeptide, and diblock copolymer, where the hydrophobic drug, additive and diblock copolymer are described herein, with an additional processing solvent; and removing the processing solvent by evaporation or distillation. The processing solvent may include an organic solvent, such as, e.g., tetrahydrofuran, ethanol, acetonitrile, chloroform, or dichloromethane. The method may further include adding water to the mixture.

In one aspect, a method is provided for preventing a disease in a mammal in need of treatment. The method includes administering an effective amount of a composition in accordance with the invention to the mammal, where the composition includes a drug that is efficacious at preventing the disease. In another aspect, a method is provided for treating a disease in a mammal in need of treatment. The method includes administering an effective amount of a composition in accordance with the invention to the mammal, where the composition includes a drug that is efficacious at preventing the disease. Optionally, the composition may include aqueous media, and also optionally the composition may include micelles. The aqueous media may include, water, deionized water, and water for injection, and optionally may also include one or more types of salts (e.g., buffer salt or NaCl) and dextrose.

The disease may be, e.g., an inflammatory conditions, autoimmune, neurological disorders, cancer, or benign hyperproliferative disease. Representative examples of diseases include arthritis, multiple sclerosis, Alzheimer's disease, psoriasis, cancer, stenosis or restenosis, benign hyperplasia (such as, e.g., hyperplasia is induced by a foreign body), cardiovascular disease, or Inflammatory Bowel Disease.

The composition may be administered by a route selected from intravenous, intraarticular, intracutaneous, interstitial, subcutaneous, intramuscular injection, insertion into the rectum, oral, and implant into the body. In one aspect, the composition is administered by intravenous delivery of an aqueous micelle solution. In another aspect, the composition is administered by implanting a solid composition in the body, where the solid composition delivers drug to the body. In yet another aspect, the composition delivers paclitaxel or an analogue or derivative thereof to the body of the mammal.

The present invention further provides a method for enhancing the rate of dissolution of a water-soluble composition that includes a hydrophobic drug and a polymer. The method includes adding an amino acid and/or an oligopeptide additive having a water-solubility of greater than about 2.5 g per 100 g water at 25° C. to the composition.

These and other aspects of the present invention will become evident upon reference to the following detailed description. In addition, various references are set forth herein. These references include the following: U.S. Patent Application Nos. 60/032,215; 60/063,087; 60/275,725; 60/288,017; 60/337,935; Ser. Nos. 08/094,536; 08/417,160; 08/480,260; 08/486,867; 08/980,549; 08/984,258; 09/013,765; 09/088,546; 09/368,871; 09/201,695; 09/294,458; and 09/368,463; U.S. Pat. Nos. 5,716,981; 5,886,026; and 5,994,341; European Patent Application Nos. 96119361.2; 97945697.7; 00123557.1; 00123534.0; and 00123537.3; European Patent No. 0 706 376; and PCT Patent Application Nos. PCT/CA94/003 73; PCT/CA97/00910; and PCT/CA99/00464.

### BRIEF DESCRIPTION OF THE DRAWINGS

FIG. 1 shows the dissolution time of polymer matrix after adding 55 mg of amino acid.

FIG. 2 shows a comparison of dissolution time with different amounts of amino acids.

FIG. 3 shows the dissolution time of polymer matrix after adding various amounts of L-phenylalanine.

FIG. 4 shows a comparison of dissolution time with 25 mg of different amino acids in the presence of MePEG.

FIG. 5 shows a comparison of dissolution time with 40 mg of different amino acids in the presence of MePEG.

### DETAILED DESCRIPTION OF THE INVENTION

In one aspect, the present invention provides hydrophobic drug-containing compositions that, upon combination with aqueous media, e.g., pure water or aqueous buffer, provide a micelle-containing composition. The hydrophobic drug, which is not very soluble in water alone, is effectively solubilized in a micelle-containing composition according to the present invention. These micelle-containing compositions contain an amphiphilic polymer that can form micelles in an aqueous solution. These compositions contain an amino acid (i.e., one or more amino acids) and/or an oligopeptide (i.e., one or more oligopeptides) additive, which assists in the solubilization of the hydrophobic drug/amphiphilic polymer composition.

For instance, in one aspect the present invention provides a composition that includes: (a) a micelle-forming biocompatible diblock copolymer (X—Y) having a hydrophilic block X comprising residues of monomer x, and a hydrophobic block Y comprising residues of monomer y; (b) an additive selected from amino acid and oligopeptide; and (c) a hydrophobic drug. The composition forms a micelle-containing solution, also known as a micellar solution, when combined with aqueous media. The composition preferably does not form any hydrogel when combined with aqueous media.

In general, the term “includes” as used above and elsewhere herein is intended to denote that the composition may, but need not, contain components not within the scope of the specifically enumerated components, which in the case of the above-described composition are components (a), (b), and (c). In various additional aspects of the present invention, the term “includes” when used herein to describe a composition may be replaced with the term “includes only”, where the term “includes only” is intended to denote that the composition contains only the enumerated components and no other components.

It should be understood that the terms “a” and “an” as used above and elsewhere herein refer to “one or more” of the enumerated components. Thus, the composition described above is intended to describe compositions that contain one or more chemically distinct diblock copolymers and one or more chemically distinct additives and one or more hydrophobic drugs.

Any concentration or other numerical ranges recited herein are to be understood to include concentrations of any integer within the range and fractions thereof, such as one tenth and one hundredth of an integer, unless otherwise indicated. It should be understood that the terms “a” and “an” as used above and elsewhere herein refer to “one or more” of the enumerated components. As used herein, the term “about” means±10% of an indicated value.

Another specific example of a composition of the present invention is a composition that includes: (a) a micelle-forming biocompatible diblock copolymer (X—Y) having a hydrophilic block X comprising residues of monomer x, and a hydrophobic block Y comprising residues of monomer y; (b) an additive which is an amino acid; and (c) a hydrophobic drug. In one aspect, the composition forms a micellar solution when combined with aqueous media. The composition preferably does not form any hydrogel when combined with aqueous media.

As another specific example, the present invention provides a composition that includes: (a) a biocompatible diblock copolymer (X—Y) having a block X comprising residues of monomer x, and a block Y comprising residues of monomer y, the block X being more hydrophilic than the block Y; (b) an additive selected from amino acid and oligopeptide; and (c) a hydrophobic drug. In one aspect, the composition forms a micellar solution when combined with aqueous media. The composition preferably does not form any hydrogel when combined with aqueous media.

As used herein, the term “comprises residues of monomer x” is used in the context of describing a polymer or copolymer. As is well known to one of ordinary skill in the art, polymers and copolymers are typically formed by the polymerization of monomers. In the formation of a polymer or copolymer, a monomer will react with a growing polymer or copolymer chain, so as to both join to the chain and also form a reactive site to which the next monomer may join. As this process repetitively occurs, the polymer or copolymer chain grows to its final length. The monomer is structurally changed by its incorporation into a polymer or copolymer, and the resulting structure is referred to herein as the residue of the monomer. Thus, a polymer or copolymer may be viewed as a chain of monomer residues.

As used herein, a “block” copolymer is a copolymer having distinct structural regions, i.e., subunit regions that are structurally distinct from one another. A subunit region is a series (chain) of monomer residues, as defined above. A diblock copolymer has two distinct structural regions, where the subunit composition in one block differs from the subunit composition in the second block. For instance, a diblock copolymer may be formed from a block (i.e., a series or chain) of acrylic acid residues adjacent to a block of methacrylic acid residues. Another example of a diblock copolymer is a block of acrylic acid residues adjacent to a block formed by a mixture of ethylene oxide and propylene oxide residues. Yet another example of a diblock copolymer is a block of residues of ethylene oxide and a block of residues from D,L-lactide.

Another specific example of a composition in accordance with the invention is a composition that includes: (a) a micelle-forming biocompatible triblock copolymer, X—Y—X or Y—X—Y, having a hydrophilic block X comprising residues of monomer x, and a hydrophobic block Y comprising residues of monomer y; (b) an additive which is an amino acid; and (c) a hydrophobic drug. In one aspect, the composition forms a micellar solution when combined with aqueous media. The composition preferably does not form any hydrogel when combined with aqueous media.

Yet another specific example of a composition in accordance with the invention is a composition that includes: (a) a biocompatible triblock copolymer, X—Y—X or Y—X—Y, having a block X comprising residues of monomer x, and a block Y comprising residues of monomer y, the block X being more hydrophilic than the block Y; (b) an additive selected from amino acid and oligopeptide; and (c) a hydrophobic drug. In one aspect, the composition forms a micellar solution when combined with aqueous media. The composition preferably does not form any hydrogel when combined with aqueous media.

The block copolymers utilized in the invention will preferably form micelles in isotonic aqueous solutions at a physiological temperature, and the micelles will have diameters within the range of about 1 nm to about 100 nm. In various embodiments, the micelles have an average diameter of 1-100, 1-90, or 1-80, or 1-70, or 1-60, or 1-50, or 1-40, or 1-30, or 1-20, or 5-100, or 5-90, or 5-80, or 5-70, or 5-60, or 5-50, or 5-40, or 5-30, or 5-20, or 10-100, or 10-90, or 10-80, or 10-70, or 10-60, or 10-50, or 10-40, or 10-30, or 10-20 nm. In a preferred embodiment, the micelles have an average diameter of about 15 nm. The copolymer blocks have “hydrophobic” and “hydrophilic” characters that are sufficiently hydrophobic and hydrophilic, respectively, to provide an amphiphilic molecule that can form a micelle in an aqueous media.

The term “biocompatible” is commonly used in the art, and is used herein according to its art-recognized meaning. For further clarity, it can be noted that a “biocompatible” material is one that does not illicit undue toxicity, irritancy, foreign body response or inflammation when it is contacted with an animal. If the biocompatible material degrades in the host, the degradation products are biocompatible degradation products.

The block copolymer is not only preferably biocompatible, but it is also preferably biodegradable. Thus, in one aspect of the invention, the block copolymer is both biocompatible and biodegradable.

As used herein, the term “micelle” has its ordinary and accustomed meaning as understood by one of ordinary skill in the art, and thus refers to a noncovalently associated collection (aggregate) of many simple molecules that together function as a unit having unique properties (e.g., aqueous solubilization of water-insoluble materials) that are not observed with the individual molecules which comprise the micelle. The micelles of the present invention are supramolecular complexes that include block copolymers, where the micelles form in aqueous solutions due to microphase separation of the nonpolar portions of the copolymers. Micelles form when the concentration of the block copolymer reaches, for a given temperature, a critical micelle concentration (CMC) that is characteristic of the copolymer. As referred to herein, a micelle is not necessarily spherical, but may assume other shapes, e.g., rod-shaped or laminar.

By varying the sizes of the hydrophilic and hydrophobic segments of the block copolymers, the tendency of the copolymers to form micelles at physiological conditions, as well as the average size of the micelles formed at the physiological conditions, can be varied. These tendencies can also be influenced by blending copolymers with differing mixes of hydrophobic and hydrophilic blocks. The micelles have a dense core formed by the water-insoluble repeating units of the Y blocks and lipophilic portions of a biological agent dissolved therein, and a hydrophilic shell formed by the X blocks and hydrophilic portions (if any) of the biological agent. The micelles have translational and rotational freedom in aqueous environment, and aqueous environments containing the micelles have low viscosity similar to water. Micelle formation typically occurs at copolymer concentrations from about 0.001 to 5% (w/v), or 0.001 to 1% (w/v), or about 0.005 to 0.5% (w/v). Here and elsewhere herein the term x % (w/v) indicates a weight of copolymer as measured in grams per volume of aqueous solution as measured in a unit of 100 milliliters. Thus, 1% (w/v) refers to 1 gram copolymer dissolved in 100 mL of solvent.

In the compositions of the present invention, the block X of the block copolymer includes residues of one or more monomers selected from (meth)acrylic acid, vinylpyrrolidone, saccharide, styrene sulfonate, 2-acrylamido-2-methyl propane sulfonic acid, acrylamide and amino acid. As used herein, (meth)acrylic acid refers both to acrylic acid and methacrylic acid. Suitable saccharides that may be a monomer for block X include, without limitation, mono-, di- and trisaccharides. Examples of saccharides are glucose, mannose, fructose, sucrose, lactose, maltose, trehalose and raffinose. Amino acids have both an amine group and a carboxylic acid group, where suitable amino acids that may be a monomer for block X include, without limitation, naturally and non-naturally occurring amino acids. Examples of naturally occurring amino acids for use in the present invention are alanine, arginine, asparagine, aspartic acid, citrulline, cysteine, cystine, glutamine, glycine, histidine, isoleucine, leucine, lysine, methionine, ornithine, phyenylalanine, proline, serine, threonine, tryptophen, tyrosine, valine, hydroxy proline, γ-carboxyglutamate, phenylglycine, or O-phosphoserine. Examples of non-naturally occurring amino acids for use in the present invention are α-alanine, α-amino butyric acid, γ-amino butyric acid, γ-(aminophenyl) butyric acid, α-amino isobutyric acid, 1-amino caproic acid, 7-amino heptanoic acid, 1-aspartic acid, aminobenzoic acid, aminophenyl acetic acid, aminophenyl butyric acid, γ-glutamic acid, cysteine (ACM), ε-lysine, ε-lysine, (A-Fmoc), methionine sulfone, norleucine, norvaline, omithine, d-omithine, p-nitro-phenylalanine, hydroxy proline, 1,2,3,4,-tetrahydroisoquinoline-3-carboxylic acid and thioproline.

In one aspect of the invention, block X of the block copolymer includes residues of alkylene oxide. Suitable alkylene oxides include ethylene oxide, propylene oxide and butylene oxide.

In another aspect, the block copolymer includes adjacent repeating units of the residue from alkylene oxide(s), so that block X comprises poly(alkylene oxide) as the sole polymeric material within block X. In another embodiment, the poly(alkylene oxide) is selected from poly(ethylene oxide) and terminal C₁-C₆ alkyl ethers of poly(ethylene oxide). As used herein, the term “C₁-C₆” refers to a moiety having from 1 to 6 carbons, i.e., having 1, 2, 3, 4, 5 or 6 carbons. A preferred terminal C₁-C₆ alkyl ether of poly(ethylene oxide) is a C₁ alkyl ether of poly(ethylene oxide), also known as methoxy polyethylene glycol (MePEG). A preferred X block of the present invention contains MePEG.

In one aspect, the block Y includes residues of monomers selected from methacrylic acid, esters of methacrylic acid, esters of acrylic acid, styrene and vinyl acetate. Suitable esters include alkyl esters (e.g., methyl ester, ethyl ester, propyl ester). In another aspect, the block Y includes residues of monomers selected from lactic acid and reactive equivalents thereof, glycolic acid and reactive equivalents thereof, and caprylic acid and reactive equivalents thereof. Reactive equivalents of lactic acid include, e.g., D-lactic acid, L-lactic acid, DL-lactic acid, DL-lactide, L-lactide and D-lactide. Reactive equivalents of glycolic acid include, e.g., glycolide. Reactive equivalents of caprylic acid include, e.g., caprolactone, valerolactone, and butyrolactone.

In another aspect, the block Y includes residues of monomers selected from trimethylene carbonate, 1,4-dioxane-2-one and 1,5-dioxepan-2one.

In one aspect of the present invention, the block Y is selected from polylactide, polyglycolide, polycaprolactone, hydrophobic polypeptides, hydrophobic polycarbonates, poly(vinyl acetate) and copolymers thereof. In one aspect, the block Y of the copolymer is poly-DL-lactide-co-glycolide, while in another aspect the block Y is poly-DL-lactide.

In one aspect, the block X includes residues of monomers selected from alkylene oxide, acrylic acid, vinyl pyrrolidone, saccharide, and amino acid, while the block Y includes residues of monomers selected from lactide or reactive equivalents thereof, glycolide or reactive equivalents thereof, caprolactone or reactive equivalents thereof trimethylene carbonate, 1,4-dioxane-2-one, 1,5-dioxepan-2one, hydrophobic amino acid, carbonate, and vinyl acetate. In one embodiment, the block X includes residues of monomers selected from alkylene oxide, while the block Y includes residues of monomers selected from lactide or reactive equivalents thereof, glycolide or reactive equivalents thereof, caprolactone or reactive equivalents thereof trimethylene carbonate, 1,4-dioxane-2-one or 1,5-dioxepan-2one. For example, block X may include residues of monomers selected from alkylene oxide(s) while Y may include residues of monomers selected from lactide or reactive equivalents thereof and glycolide or reactive equivalents thereof. As another example, block X includes residues of ethylene oxide while block Y comprises residues of lactide. As yet another example, block X is MePEG and block Y is poly(DL-lactide).

The relative amounts, on a weight basis, of the blocks X and Y in the diblock copolymer is preferably controlled to allow the block copolymer to form a micelle in aqueous media. In one aspect, 100 parts of block copolymer includes 30-90 parts hydrophilic polymer X and 70-10 parts hydrophobic polymer Y, where these parts are on a weight basis. In another aspect, 100 parts of block copolymer include 40-80 parts hydrophilic polymer X and 60-20 parts hydrophobic polymer Y. In yet another aspect, 100 parts of block copolymer include 50-70 parts hydrophilic polymer X and 50-30 parts hydrophobic polymer Y. In still another aspect, 100 parts of block copolymer include about 60 parts hydrophilic polymer X and about 40 parts hydrophobic polymer Y.

The relative amounts, on a weight basis, of the blocks X and Y in the block copolymer can be controlled in the following manner: a stoichiometric ratio of X:Y, x:y, X:y or x:Y may be combined as reagents in the reaction to produce the diblock copolymer. The result is a polymer having the composition of X:Y. See, e.g., Zhang, X. et al. International Journal of Pharmaceutics 132:195-206 (1996).

The molecular weight of the block copolymer, in terms of number average molecular weight, is preferably controlled in order that the block copolymer may form a micelle in aqueous media. In one aspect, the block copolymer has a number average molecular weight of about 1,000 to about 10,000 g/mol. In another aspect, the block copolymer has a number average molecular weight of about 2,000 to about 5,000 g/mol. In yet another aspect, the block copolymer has a number average molecular weight of about 2,500 to about 3,500 g/mol.

The molecular weight of the block copolymer containing the blocks X and Y can be controlled by selecting appropriate reaction conditions. By example, for the preparation of a diblock copolymer wherein X is polyalkylene oxide (from MePEG) and Y is poly-DL-lactide, the molecular weight is controlled by selecting a specific molecular weight of MePEG (block X) as a starting material and a specific ratio of X:y (where y is DL-lactide, the other starting material). In this synthesis the molecular weight is expected to be equal to:

diblock copolymer molecular weight=molecular weight of X+mass of y/mass of X*molecular weight of X

See, e.g., Zhang, X. et al. International Journal of pharmaceutics 132:195-206 (1996).

Other copolymers that can be used in the instant compositions include those that are described in U.S. Pat. Nos. 6,616,941; 6,599,519; 6,410,057; 6,322,805; 6,267,987; 6,210,717 and PCT Publication Nos. WO 03/033592; WO 03/033592; WO 03/000778; WO 02/45689; WO 02/072150; WO 01/97611; WO 01/87345; WO 01/85216; WO 01/12718; WO 01/05379; and WO 97/10849.

In one aspect of the invention, the block copolymers can be purified following synthesis and prior to final use. Typically, after preparation of the diblock copolymer, the product mixture will contain some unreacted starting materials and/or some by-products, i.e., reaction products that are other than the desired reaction products. In the synthesis of diblock copolymer comprising a methoxypolyethylene glycol block and a poly(DL-lactide) block, the by-products and/or unreacted starting material(s) may be residual monomer and other components that are acidic in nature. In this polymer the residual monomer is DL-lactide. For diblock copolymers in which the hydrophobic block is a polyester, acidic by-products are anticipated to form, however their exact composition will vary depending on the monomer used.

Purification methods are described, for example, in WO 02/072150. In one aspect, the diblock copolymer used in forming the final composition of the invention is 80% pure, which means that in one aspect the invention provides a composition that includes a diblock copolymer where at least 80% of the weight of the composition is contributed by the diblock copolymer. In other aspects of the invention, the diblock copolymer is 85% pure, 90% pure, 92% pure, 94% pure, 95% pure, 96% pure, 97% pure, 98% pure, 99% pure, 99.5% pure, or 99.9% pure.

A diblock copolymer useful in forming micelles according to the present invention may be synthesized according to methods disclosed in publications such as Zhang et al, 1996. Whether formed according to Zhang et al. or by some other procedure, the diblock copolymer may, as part of the synthesis process or at some later time, be exposed to organic solvents and/or activated carbon. It will typically be desirable to separate the diblock copolymer from the organic solvents and/or activated carbon. In one aspect of the invention, the diblock copolymer, as well as any starting materials and/or by-products that are in admixture with the diblock copolymer, is dissolved in an organic solvent and combined with activated carbon (also referred to herein as activated charcoal). After the activated carbon has had an opportunity to interact with the copolymer-containing composition, which occurs in a timeframe on the order or 10-60 minutes, the carbon is removed from the copolymer in a manner that allows starting materials and/or by-products that interact with the carbon, to be removed with the carbon.

For example, the copolymer may be dissolved in an organic purification solvent, where dissolution may involve heating up to about 55° C. The copolymer concentration in the solvent may be up to 50% on a weight basis, but is preferably less than 10%, less than 5% or less than or equal to 2.5%. The organic solvent may contain small amounts of water, preferably less than 20%, less than 10%, less than 5% or less than 2% of the total solvent volume. Suitable organic purification solvents include, but are not limited to, dichloromethane, ethanol, isopropanol, tetrahydrofuran or chloroform. Activated charcoal is added to this solution with mixing. After mixing, the activated charcoal is removed by means such as centrifugation or filtration. The resulting solution is then subjected to means that remove the solvent from the copolymer. For instance, the solvent may be removed by means such as drying under increased heat, and/or under vacuum or forced air or a dry gas such as nitrogen. Spray drying and freeze drying are two solvent-removal methods according to the present invention. Labconco, Kansas City, Mich., is one supplier of freeze dryer systems and solvent evaporation systems. The use of a vacuum oven is a preferred option for removing solvent, where numerous suppliers of vacuum ovens are known to one of ordinary skill in the art, see, e.g., Binder GmbH, Germany; and M. Braun Inc., Stratham, N.H.

As another example, the copolymer may be dissolved in an organic solvent where the solvent and copolymer/solvent concentration are selected such that the copolymer is soluble in the solvent at elevated temperatures, but insoluble or partially insoluble at reduced temperatures. In other words, the copolymer can be crystallized or precipitated from the solvent. Isopropanol is a suitable solvent for this purpose. Mixing the copolymer with the solvent may involve heating in order to facilitate dissolution of the copolymer. In the case of isopropanol, a temperature of up to about 55° C. is suitable. After mixing, the solution is cooled to facilitate precipitation of the copolymer from the solvent. The cooling temperature may be as low as about −20° C., but temperatures as high as 2-8° C. are suitable for some solvents, such as isopropanol. After precipitation, the copolymer can be isolated from the solvent by, for example, filtration, and further dried if necessary by, for example, exposure to reduced pressure and/or elevated temperature that will encourage evaporation of the solvent. This process may be repeated as many times as necessary to achieve a copolymer with the desired properties. In one aspect, the process is repeated once. In another aspect the process is repeated twice.

The desirability of removing unreacted starting materials and/or acidic reaction by-products can be seen by reference to the data in Table 1. The data in Table 1 demonstrate that when acidic oligomers of DL-lactide and/or DL-lactide itself are added to a diblock copolymer or polyethylene glycol polymer containing little or none of these components, polymer matrices are produced that incorporate paclitaxel into the matrix with varying amounts of paclitaxel loss in the process. Addition of paclitaxel to the polymer matrices resulted in a reduction of paclitaxel content from the amount added and these reductions correspond to matrices containing elevated levels of the acidic species and residual monomer. According to the data, when paclitaxel is combined with a diblock copolymer having up to 2% residual monomer and up to 0.2 μmol/mg acid content (expressed as the number of protons titrated in a solution prepared by dissolving 1 mg of diblock copolymer in water), after heating, only 95% of the paclitaxel initially added to the mixture could be recovered, as quantified using a reverse phase HPLC assay with UV detection and a taxane optimized C18 column. Furthermore, in mixtures in which the diblock copolymer had greatly reduced quantities of the residual monomer and acidic components, 98% of the paclitaxel could be recovered and when paclitaxel was combined with methoxypolyethylene glycol having no measured acidic components and no DL-lactide monomer, 99% was recovered. Addition of DL-lactide and an acidic component resulted in further loss, compared to samples to which neither of these components were added.

TABLE 1
RECOVERY OF PACLITAXEL ADDED TO SEVEN
DIFFERENT MIXTURES AFTER HEATING EACH
OF THE MIXTURES TO 55° C. FOR 1 HOUR

| | Mixture number | | | | | | |
|---|---|---|---|---|---|---|---|
| | (X denotes the | | | | | | |
| | component is | | | | | | |
| Selected components | present in the mixture) | | | | | | |
| contained in the mixture | 1 | 2 | 3 | 4 | 5 | 6 | 7 |
| Methoxy polyethylene glycol | | X | X | | | | |
| Diblock copolymer | | | X | | | | |
| (not more than 2% residual monomer | | | | | | | |
| and 0.2 μmol of protons/mg in | | | | | | | |
| aqueous solution) | | | | | | | |
| Diblock copolymer | | | | X | X | X | X |
| (less than 0.5% residual | | | | | | | |
| monomer and 0.05 μmol | | | | | | | |
| of protons/mg in | | | | | | | |
| aqueous solution) | | | | | | | |
| DL-lactide monomer (2%) | | | | | X | | X |
| DL-lactide oligomer | | | | | | X | X |
| (having 4 μmol of | | | | | | | |
| protons/mg in | | | | | | | |
| aqueous solution) | | | | | | | |
| Recovery of paclitaxel(% of | 100 | 99 | 95 | 98 | 95 | 95 | 96 |
| after heating amount | | | | | | | |
| added to mixture) | | | | | | | |

Based on these data, preferred diblock copolymers according to the present invention have the following compositional restrictions. In one aspect, the diblock copolymers comprising a polyalkylene oxide block and a polyester block have less than 5% residual monomer (from polyester starting material) and less than 0.4 μmol/mg acid (by an aqueous titration method). More preferred are limits of 2% residual monomer and 0.2 μmol/mg acid. Even more preferred are limits of 1% residual monomer and 0.05 μmol/mg acid. Still more preferred are limits of 0.5% residual monomer and 0.025 μmol/mg acid. The disclosed limits of these two components may be applied independent of one another. For instance, in one aspect the diblock copolymer is in combination with less than 5% residue monomer, and less than 0.025 μmol/mg acid. Every other combination of % residue monomer and upper limit of μmol/mg acid value as set forth above are provided according to various aspects of the present invention.

In another aspect of purification, precipitation from a solvent can lighten the color of the copolymer as a result of removing constituents and/or byproducts of the reaction which absorb light in the range of 300 to 500 nm, with a maximum absorbance at 315 nm, and a significant absorbance in many batches of diblock copolymer at 450 nm. After purification the copolymer is characterized by absorbance characteristics in the Table 2.

TABLE 2
LIGHTENING COLOR OF DIBLOCK COPOLYMER

| | Absorbance | | |
|---|---|---|---|
| | Values (AU) At: | | |
| Batch # | 315 nm | 425 nm | 450 nm |
| 1 unpurified | 2.58 | 0.464 | 0.262 |
| 1 purified | 0.442 | 0.137 | 0.100 |
| 2 unpurified | 2.39 | 0.264 | 0.136 |
| 2 purified | 0.178 | 0.0438 | 0.0330 |
| 3 unpurified | 1.24 | 0.0856 | 0.0496 |
| 3 purified | 0.0912 | 0.0274 | 0.0222 |

In these aspects, in which purification results in a lighter product, the change in color may also be assessed by such suitable methods as ASTM method D1209. In Table 2, samples were prepared by dissolving 675 mg of sample in 5 mL of phosphate buffer, and then UV absorbance measurements were taken.

In some aspects, the copolymer may contain constituents and/or byproducts which absorb at 315 nm but with an intensity such that the absorbance at 450 nm does not result in a visible yellow color. In these aspects, the purification method is suitable in removing the constituents such that absorbance at 315 nm is suitably reduced, from values in the range of 0.6 to 1.7 AU to values less than 0.1 AU.

Thus, in one aspect of the invention, a purification process is provided whereby the diblock copolymer is provided having a lighter color, e.g., a reduced yellowness, compared to the color of the starting diblock copolymer. The purification process entails precipitation of the copolymer and/or contact of a solution containing the copolymer with activated charcoal, both as described herein, in order to achieve a less intensely colored diblock copolymer. Purification of the diblock copolymer may be affected either before or after incorporating the additive into the composition, provided the additive and the copolymer will both precipitate. An example of such an additive is L-phenylalanine.

The compositions of the present invention contain an additive. The additive may also be referred to as a solubilizing additive because one of its key functions is to assist in the solubilization of the components when that composition is combined with aqueous media. When present, the additive imparts advantageous properties to the composition. For example, the additive-containing compositions of the present invention are particularly advantageous in that they form micelles at an enhanced rate, have an enhanced ability to incorporate drug(s); and/or have advantageous physical characteristics.

In one aspect of the present invention the additive is an amino acid. In one aspect the amino acid is one of the naturally-occurring amino acids, e.g., L-alanine, L-arginine, L-asparagine, L-aspartic acid, L-cysteine, L-glutamine, L-glutamine acid, glycine, L-histidine, L-isoleucine, L-leucine, L-lysine, L-methionine, L-phenylalanine, L-proline, L-serine, L-threonine, L-tryptophane, L-tyrosine, and L-valine. In another aspect, the amino acid is the unnatural stereoisomer of a naturally occurring amino acid, e.g., D-alanine, D-arginine, D-asparagine, D-aspartic acid, D-cysteine, D-glutamine, D-glutamine acid, D-histidine, D-isoleucine, D-leucine, D-lysine, D-methionine, D-phenylalanine, D-proline, D-serine, D-threonine, D-tryptophane, D-tyrosine, and D-valine.

In another aspect of the invention the additive is an oligopeptide. As used herein, the term “oligopeptide” refers to a short polypeptide, i.e., a poly(amino acid) having 2-10 amino acid residues. The amino acid residues present in the oligopeptide may be identical to one another, or not. In some embodiments, e.g., the same type of amino acid residue is present throughout the oligopeptide. For example, an oligopeptide may contain only L-alanine residues. In this case, the oligopeptide is referred to as an oligo(L-alanine). In other embodiments, the oligopeptide may contain two or more different amino acid residues, such as, for example, phenylalanine-serine, phenylalanine-valine, and the like.

The dissolution rate of the polymer/hydrophobic drug combination may be enhanced by including in the composition an additive that contains an amino acid or oligopeptide having a solubility of greater than about 2.5 g/100 g water at 25° C. Examples of such amino acids include the L and D isomers or alanine, arginine, asparagine, cysteine, glutamine, glycine, histidine, isoleucine, lysine, methionine, phenylalanine, proline, threonine, and valine.

The composition may, in addition to the additive, contain a co-additive. The co-additive may be a polymer, which may be either hydrophilic or hydrophobic. In one aspect, the co-additive polymer is hydrophilic. Optionally, the co-additive hydrophilic polymer has a molecular weight of 200-5,000. Optionally, the co-additive hydrophilic polymer is selected from poly(ethylene oxide) and the terminal C₁-C₆ alkyl ethers thereof. In a preferred embodiment, the co-additive polymer is MePEG. When the co-additive is MePEG, in one aspect, the MePEG has a molecular weight of 200-750 g/mol, while in another aspect the MePEG has a molecular weight of 550-2000 g/mol, while in yet another aspect the MePEG has a molecular weight of 750-5000 g/mol, where these molecular weight ranges are set forth in terms of number average molecular weight. Alternatively, the co-additive polymer may be hydrophobic. A suitable hydrophobic co-additive polymer is poly(DL-lactide). In one aspect, the hydrophobic co-additive polymer has a number average molecular weight of 288 to about 1,000.

In addition to the additive, and optionally in addition to the co-additive, the compositions of the present invention may include an organic solvent, which will be referred to herein as the additive solvent. Preferably, the additive solvent is biocompatible. The additive solvent should be biocompatible because it will be administered to the subject along with the drug and diblock copolymer. In one aspect, the additive solvent is N-methyl-2-pyrrolidone (NMP). Other examples of biocompatible additive organic solvents include PEG 200, propylene glycol, dimethylsulfoxide (DMSO), ethanol, ethoxydiglycol. Analogues and homologues of any of the above-identified solvents also may be suitable for use in the instant compositions.

The compositions and methods of the present invention include a drug. Drugs includes those therapeutic and prophylactic agents which may mitigate, treat, cure or prevent a given disease or condition. Representative examples of drug classes and drugs are discussed in more detail below, and include, for example, chemotherapeutic, antibiotic, antimicrotubule, analgesics, anthelmintics, anti-arrhythmic agents, anesthetics, anti-thrombotic, vasodialators, prostoglandins, vitamins, psychotherapeutic agents, anti-bacterial, anti-coagulants, anti-depressants, anti-diabetics, anti-epileptics, anti-fungal agents, anti-hypertensive agents, anti-malarials, anti-migraine agents, immunosuppressants, anti-protazoal agents, sedatives, beta.-blockers, corticosteroids, diuretics, lipid regulating agents, anti-anginal agents, hormones, anti-inflammatory, and antiproliferative agents.

In particular, the inventive compositions and methods include a hydrophobic drug. The term “hydrophobic drug” refers to a drug that is insoluble or sparingly or poorly soluble in water. As used herein, such drugs have a solubility below 10 mg/ml, usually below 1 mg/ml, sometimes below 0.01 mg/ml, and sometimes below 0.001 mg/ml. Typically, hydrophobic drugs are difficult to incorporate into aqueous delivery vehicles because of their limited solubility in water. The present invention provides rapidly dissolving micelle-forming compositions, and micellar compositions, and methods of making and using same, that include the hydrophobic drug.

Exemplary hydrophobic drugs include but are not limited to the following drug classes and examples: analgesics and anti-inflammatory agents, such as aloxiprin, auranofin, azapropazone, benorylate, diflunisal, etodolac, fenbufen, fenoprofen calcium, flurbiprofen, ibuprofen, indomethacin, ketoprofen, meclofenamic acid, mefenamic acid, nabumetone, naproxen, oxyphenbutazone, phenylbutazone, piroxicam, and sulindac; anthelmintics, such as albendazole, bephenium hydroxynaphthoate, cambendazole, dichlorophen, ivermectin, mebendazole, oxamniquine, oxfendazole, oxantel embonate, praziquantel, pyrantel embonate, and thiabendazole; anti-arrhythmic agents, such as amiodarone HCl, disopyramide, flecainide acetate, and quinidine sulphate; anti-bacterial agents, such as benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, and trimethoprim; anti-coagulants, such as dicoumarol, dipyridamole, nicoumalone, and phenindione; anti-depressants, such as amoxapine, maprotiline HCl, mianserin HCl, nortriptyline HCl, trazodone HCl, and trimipramine maleate; anti-diabetics, such as acetohexamide, chlorpropamide, glibenclamide, gliclazide, glipizide, tolazamide, and tolbutamide; anti-epileptics, such as beclamide, carbamazepine, clonazepam, ethotoin, methoin, methsuximide, methylphenobarbitone, oxcarbazepine, paramethadione, phenacemide, phenobarbitone, phenyloin, phensuximide, primidone, sulthiame, and valproic acid; anti-fungal agents, such as amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine HCl, terconazole, tioconazole, and undecenoic acid; anti-gout agents, such as allopurinol, probenecid, and sulphin-pyrazone; anti-hypertensive agents, such as amlodipine, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCl, reserpine, and terazosin HCl; anti-malarials, such as amodiaquine, chloroquine, chlorproguanil HCl, halofantrine HCl, mefloquine HCl, proguanil HCl, pyrimethamine, and quinine sulphate; anti-migraine agents, such as dihydroergotamine mesylate, ergotamine tartrate, methysergide maleate, pizotifen maleate, and sumatriptan succinate; anti-muscarinic agents, such as atropine, benzhexol HCl, biperiden, ethopropazine HCl, hyoscyamine, mepenzolate bromide, oxyphencylcimine HCl, and tropicamide; anti-neoplastic agents and immunosuppressants, such as aminoglutethimide, amsacrine, azathioprine, busulphan, chlorambucil, cyclosporin, sirolimus and functional analogues of sirolimus (e.g., SDZ-RAD, CCI-779, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, and everolimus), dacarbazine, estramustine, etoposide, lomustine, melphalan, mercaptopurine, methotrexate, mitomycin, mitotane, mitozantrone, procarbazine HCl, tamoxifen citrate, paclitaxel and functional paclitaxel derivatives, and testolactone; anti-protazoal agents, such as benznidazole, clioquinol, decoquinate, diiodohydroxyquinoline, diloxanide furoate, dinitolmide, furzolidone, metronidazole, nimorazole, nitrofurazone, ornidazole, and tinidazole; anti-thyroid agents, such as carbimazole and propylthiouracil; anxiolytic, sedatives, hypnotics and neuroleptics, such as alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, and zopiclone; beta.-blockers, such as acebutolol, alprenolol, atenolol, labetalol, metoprolol, nadolol, oxprenolol, pindolol, and propranolol; cardiac inotropic agents, such as amrinone, digitoxin, digoxin, enoximone, lanatoside C, and medigoxin; corticosteroids, such as beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, and triamcinolone; diuretics, such as acetazolamide, amiloride, bendrofluazide, bumetanide, chlorothiazide, chlorthalidone, ethacrynic acid, frusemide, metolazone, spironolactone, and triamterene; anti-parkinsonian agents, such as bromocriptine mesylate, and lysuride maleate; gastro-intestinal agents, such as bisacodyl, cimetidine, cisapride, diphenoxylate HCl, domperidone, famotidine, loperamide, mesalazine, nizatidine, omeprazole, ondansetron HCl, ranitidine HCl, and sulphasalazine; Histamine H,-Receptor Antagonists, such as acrivastine, astemizole, cinnarizine, cyclizine, cyproheptadine HCl, dimenhydrinate, flunarizine HCl, loratadine, meclozine HCl, oxatomide, and terfenadine; lipid regulating agents, such as bezafibrate, clofibrate, fenofibrate, gemfibrozil, and probucol; nitrates and other anti-anginal agents, such as amyl nitrate, glyceryl trinitrate, isosorbide dinitrate, isosorbide mononitrate, and pentaerythritol tetranitrate; nutritional agents, such as betacarotene, vitamin A, vitamin B.sub.2, vitamin D, vitamin E, and vitamin K; opioid analgesics, such as codeine, dextropropyoxyphene, diamorphine, dihydrocodeine, meptazinol, methadone, morphine, nalbuphine, and pentazocine; sex hormones, such as clomiphene citrate, danazol, ethinyl estradiol, medroxyprogesterone acetate, mestranol, methyltestosterone, norethisterone, norgestrel, estradiol, conjugated oestrogens, progesterone, stanozolol, stibestrol, testosterone, tibolone; and stimulants, such as amphetamine, dexamphetamine, dexfenfluramine, fenfluramine, and mazindol.

In one preferred aspect, the hydrophobic drug is selected from the following classes of compounds: chemotherapeutic, antibiotic, antimicrotubule, anti-inflammatory, and antiproliferative compounds. In a more preferred aspect, the hydrophobic drug is selected from paclitaxel, hydrophobic paclitaxel derivatives and hydrophobic paclitaxel analogues. In another more preferred aspect, the hydrophobic drug is paclitaxel.

Within one preferred embodiment of the invention, the hydrophobic drug is paclitaxel, a compound currently recognized to disrupt mitosis (M-phase) by binding to tubulin to form abnormal mitotic spindles or an analogue or derivative thereof. Briefly, paclitaxel is a highly derivatized diterpenoid (Wani et al., J. Am. Chem. Soc. 93:2325, 1971). It may be obtained, for example, from the harvested and dried bark of Taxus brevifolia (Pacific Yew) and Taxomyces Andreanae and Endophytic Fungus of the Pacific Yew (Stierle et al., Science 60:214-216, 1993). “Paclitaxel” as used herein refers to hydrophobic formulations including paclitaxel, prodrugs, analogues and derivatives such as, for example, TAXOL®, TAXOTERE®, docetaxel, 10-desacetyl analogues of paclitaxel and 3N-desbenzoyl-3′N-t-butoxy carbonyl analogues of paclitaxel) may be readily prepared utilizing techniques known to those skilled in the art (see, e.g., Schiff et al., Nature 277:665-667, 1979; Long and Fairchild, Cancer Research 54:4355-4361, 1994; Ringel and Horwitz, J. Nat'l Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402—from Taxus brevifolia).

Representative examples of paclitaxel derivatives and analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol (2′- and/or 7-O-ester derivatives), (2′- and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2′succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl) taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol analogues with modified phenylisoserine side chains, taxotere, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin); and other taxane analogues and derivatives, including 14-beta-hydroxy-10 deacetybaccatin III, debenzoyl-2-acyl paclitaxel derivatives, benzoate paclitaxel derivatives, phosphonooxy and carbonate paclitaxel derivatives, sulfonated 2′-acryloyltaxol; sulfonated 2′-O-acyl acid paclitaxel derivatives, 18-site-substituted paclitaxel derivatives, chlorinated paclitaxel analogues, C4 methoxy ether paclitaxel derivatives, sulfenamide taxane derivatives, brominated paclitaxel analogues, Girard taxane derivatives, nitrophenyl paclitaxel, 10-deacetylated substituted paclitaxel derivatives, 14-beta-hydroxy-10 deacetylbaccatin III taxane derivatives, C7 taxane derivatives, C10 taxane derivatives, 2-debenzoyl-2-acyl taxane derivatives, 2-debenzoyl and -2-acyl paclitaxel derivatives, taxane and baccatin III analogues bearing new C2 and C4 functional groups, n-acyl paclitaxel analogues, 10-deacetylbaccatin III and 7-protected-10-deacetylbaccatin III derivatives from 10-deacetyl taxol A, 10-deacetyl taxol B, and 10-deacetyl taxol, benzoate derivatives of taxol, 2-aroyl-4-acyl paclitaxel analogues, orthro-ester paclitaxel analogues, 2-aroyl-4-acyl paclitaxel analogues and 1-deoxy paclitaxel and 1-deoxy paclitaxel analogues.

In one aspect, the hydrophobic drug is a taxane having the formula (1):

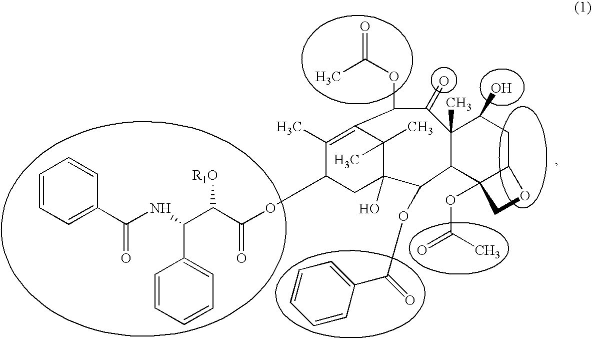

where the gray-highlighted portions may be substituted and the non-highlighted portion is the taxane core. A side-chain (labeled “A” in the diagram) is desirably present in order for the compound to have good activity as a cell cycle inhibitor. Examples of compounds having this structure include paclitaxel (Merck Index entry 7117), docetaxol (Taxotere, Merck Index entry 3458), and 3′-desphenyl-3′-(4-ntirophenyl)-N-debenzoyl-N-(t-butoxycarbonyl)-10-deacetyltaxol.

In one aspect, suitable taxanes such as paclitaxel and its hydrophobic analogues and derivatives are disclosed in U.S. Pat. No. 5,440,056 as having the structure (2):

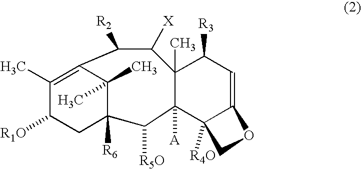

wherein X may be oxygen (paclitaxel), hydrogen (9-deoxy derivatives), thioacyl, or dihydroxylprecursors; R₁ is selected from paclitaxel or taxotere side chains or alkanoyl of the formula (3)

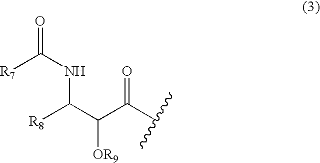

wherein R₇ is selected from hydrogen, alkyl, phenyl, alkoxy, amino, phenoxy (substituted or unsubstituted); R₈ is selected from hydrogen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydrogen, hydroxyl, alkoyl, alkanoyloxy, aminoalkanoyloxy, and peptidyalkanoyloxy; R₃ is selected from hydrogen or oxygen-containing groups, such as hydrogen, hydroxyl, alkoyl, alkanoyloxy, aminoalkanoyloxy, and peptidyalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidyalkanoyloxy.

In one aspect, the paclitaxel analogues and derivatives useful as a hydrophobic drug according to the present invention are disclosed in PCT International Patent Application No. WO 93/10076. As disclosed in this publication, the analogue or derivative should have a side chain attached to the taxane nucleus at C₁₃, as shown in the structure below (formula 4), in order to confer antitumor activity to the taxane.

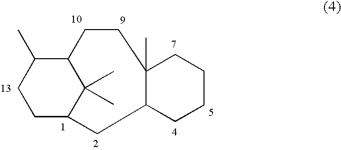

PCT International Publication No. WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, 10. As well, an oxetane ring may be attached at carbons 4 and 5. As well, an oxirane ring may be attached to the carbon labeled 4.

In one aspect, the taxane-based hydrophobic drug useful in the present invention is disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aryol, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

In another aspect, the taxane-based hydrophobic drug useful in the present invention is disclosed in U.S. Pat. No. 6,107,332.

The inventive composition may further include a buffering constituent. The buffering constituent is present so that, upon formation of an aqueous micellar solution, the solution has a specific pH. The preferred pH for said aqueous micellar solution is physiological pH. In one aspect, the buffering constituent includes a phosphate salt.

In one aspect, the present invention provides compositions that include a block copolymer, an additive selected from amino acid and oligopeptide, a hydrophobic drug (e.g., paclitaxel) and buffering constituent (e.g., phosphate salt). The following compositions are exemplary compositions of the present invention, wherein paclitaxel is identified as a particular hydrophobic drug and phosphate salt is identified as a particular buffering constituent. Thus, in one aspect, the present invention provides a composition comprising a copolymer in the range of about 10-90 percent (w/w), an additive selected from amino acid and oligopeptide in the range of about 5 to 70 percent (w/w), a hydrophobic drug in the range of 1 to 15 percent (w/w) and a buffering component in the range of 1-20 percent (w/w). In one aspect, the copolymer is a X—Y, X—Y—X or a Y—X—Y copolymer. In another aspect, the copolymer is a X—Y copolymer, such as, for example, a methoxy polyethylene glycol-co-poly(D,L-lactide) diblock copolymer.

In another aspect, the present invention provides a composition comprising about 60 percent (w/w) diblock copolymer having a weight ratio of methoxypolyethylene glycol block to poly(DL-lactide) block of about 60:40 and a molecular weight of about 3,300; about 33 percent (w/w) additive selected from amino acid and oligopeptide; about 7 percent (w/w) paclitaxel; where these percentages are based on the total weight of these three ingredients, and other components may be present in the composition.

In another aspect, the present invention provides a composition comprising about 64 percent (w/w) diblock copolymer having a weight ratio of methoxypolyethylene glycol block to poly(DL-lactide) block of about 60:40 and a molecular weight of about 3,300; about 29 percent (w/w) additive selected from amino acid and oligopeptide; about 7 percent (w/w) paclitaxel; where these percentages are based on the total weight of these three ingredients, and other components may be present in the composition.

In another aspect, the present invention provides a composition that includes about 73 percent (w/w) diblock copolymer having a weight ratio of methoxypolyethylene glycol block to poly(DL-lactide) block of about 60:40 and a molecular weight of about 3,300; about 19 percent (w/w) additive selected from amino acid and oligopeptide; about 8 percent (w/w) paclitaxel, where these percentages are based on the total weight of these three ingredients, and other components may be present in the composition.

In another aspect, the present invention provides a composition comprising about 83 percent (w/w) diblock copolymer having a weight ratio of methoxypolyethylene glycol block to poly(DL-lactide) block of about 60:40 and a molecular weight of about 3,300; about 8 percent (w/w) additive selected from amino acid and oligopeptide; about 9 percent (w/w) paclitaxel; where these percentages are based on the total weight of these three ingredients, and other components may be present in the composition.

The preparation of the non-aqueous compositions of the present invention can be accomplished in a number of ways. In one aspect, the copolymer, hydrophobic drug and amino acid or oligopeptide are dissolved in a common solvent to form a homogeneous solution. The solvent is then removed to provide a solid composition of the three components. In another aspect, the copolymer and the hydrophobic drug can be dissolved in a common solvent. The amino acid or oligopeptide can be added in the solid form to produce a non-homogeneous solution. The solvent is then removed to provide a solid composition of the three components. In another aspect, the copolymer and the hydrophobic drug can be dissolved in a common solvent. The solvent is then removed. The resultant copolymer/hydrophobic drug mixture in then constituted in an aqueous solution that contains an amino acid or oligopeptide. This aqueous solution may optionally include buffer salt. The water is then removed to provide a solid composition of at least the three components. A suitable processing solvent is tetrahydrofuran, ethanol, acetonitrile, chloroform, and/or dichloromethane, combinations of these solvents. Combinations of water with tetrahydrofuran, ethanol or acetonitrile, as well as combinations thereof, can also be used.

Solvent removal can be accomplished using elevated temperature, a flow of a gas over the surface of the composition, reduced pressure, spray drying or lyophilization as well as a combination of the above methods.

Water removal can be accomplished using elevated temperature, a flow of a gas over the surface of the composition, reduced pressure, spray drying or lyophilization as well as a combination of the above methods. Lyophilization is a preferred method for water removal. The solid composition obtained can be used in the form that it is obtained or can be further processed by milling, grinding, sieving or a combination of these processes.

Methods for preparing non-aqueous compositions are described, e.g., in WO 02/072150. In one aspect, a solvent is selected that dissolves each of paclitaxel, diblock copolymer and co-additive if present. For example, diblock copolymer and polymeric co-additive may be combined and heated to achieve a molten mixture. To this melt is then added a solution of paclitaxel in an organic solvent, preferably tetrahydrofuran. Other orders of combining the ingredients may be employed. In another suitable approach, the diblock copoymer and the drug are dissolved in a suitable solvent, then the solvent removed under vacuum or forced air. The dried residue is treated with an aqueous buffer to produce a micelle solution that is then treated with amino acid, where the amino acid is optionally pre-dissolved in water or buffer. The product is then dried, e.g., by freeze-drying. Regardless of the order of combining the ingredients, in the end a dried mixture of diblock copolymer, drug and additive (amino acid and/or oligopeptide) is prepared.

For example, about 20 mL of THF may be used to dissolve about 990 mg of diblock coplymer and 110 mg of paclitaxed. An aqueous solution of amino acids and/or oligopeptide may then be added and the volatile solvents (water and THF) removed under vacuum. Removal of the solvent may be accomplished by exposing the composition to a reduced pressure, i.e., a vacuum, and/or an elevated temperature, i.e., a temperature greater than about 23° C., and/or to a stream of dry gas, e.g., nitrogen or argon. Under conditions of reduced pressure and/or elevated temperature and/or exposure to a stream of dry gas, evaporation of the solvent is expedited. Preferably, both reduced pressure and elevated temperature are used to facilitate solvent removal. The drying process conditions of temperature and pressure and time may be varied to optimize the process for scale and equipment used. In small scale experiments, drying times range from 2 to 72 hours, temperatures range from ambient to 75° C., and gas environment ranges from forced air to reduced pressure to full vacuum. These conditions may be optimized for the scale of the reaction. The dry or nearly dry residue resulting from this process can then be ground up to provide a homogeneous powder.

One shortcoming with this approach to preparing the non-aqueous compositions is that paclitaxel tends to degrade when exposed to elevated temperature. The degradation can be mitigated by using relatively lower elevated temperature, e.g., 35° C. However, as the elevated temperature is reduced, a corresponding increase in the time to achieve the same level of solvent evaporation is typically observed. Another shortcoming with this approach to preparing the non-aqueous compositions of the invention is that residual solvent typically remains in the product. This problem may, in part, be addressed by using as little solvent as possible in the preparation of the compositions. However, minimizing the solvent can only go part way toward solving the basic problem because some amount of solvent is typically necessary for the process to work successfully. For purposes of consistency and safety, the amount of residual solvent is desirably minimized.

In order to avoid problems with residual solvent and paclitaxel degradation as discussed above, the present invention provides alternative methods of preparing the non-aqueous compositions. Thus, in one aspect, an organic solution of paclitaxel is prepared and then combined with a portion of the carrier, i.e., the diblock copolymer, and the additive. Sufficient solvent is used to just dissolve all the components so that a homogeneous solution results. Slightly elevated temperature may be, and preferably is employed to achieve a completely homogeneous solution. Of course, the elevated temperature should be below the boiling point of the solvent and the degradation temperature of the drug. After a homogeneous solution has been achieved, the majority, if not all of the solvent is removed using reduced pressure and/or elevated temperature as discussed above. The residue, which will be molten at the elevated temperature, is then combined with the remaining carrier components while maintaining a liquid state. The homogeneous mixture is then cooled to room temperature whereupon it solidifies.

Another method to prepare the water-free compositions of the present invention avoids the use of organic solvent altogether. According to this method, solid dibock copolymer and solid paclitaxel are combined and then mixed and/or milled to achieve a somewhat homogeneous mixture. This mixture is then melted to produce a substantially liquid composition. At this point the mixture may still contain some solid paclitaxel. The mixture is cooled and milled at low temperature, e.g., by contact with dry ice and the milled powder allowed to warm to room temperature. The milled powder is then heated to a molten state, typically achieved at 60-75° C. This process of melting and milling is repeated as needed until a homogeneous melt is obtained, i.e., a melt that is free of solid paclitaxel. Two cycles are typically sufficient to achieve a homogeneous melt for the composition on a 5 gram scale. Additional heating/milling cycles may be employed as the amount of the components is increased. Also, as the scale increases, stirring speed may be adjusted as needed to increase the amount of paclitaxel that dissolves in the melt. Residual materials that resist melting after a number of heating/cooling cycles may be removed by filtration of the liquid, or by sieving of the powder. The residue powder may contain particles in the micron size range. Additive, i.e., amino acid and/or oligopeptide, which is typically in a crystalline powder form, may then be combined with the residue powder and the two powders mixed well to achieve a uniform state.

In any of the compositions described above, unless water is specifically identified as being present in the composition, the compositions typically have less than 5% (w/w) moisture content or may be in an anhydrous form. In a preferred aspect, the compositions have a moisture content less than 0.5% (w/w) and have been produced through lyophilization of a micellar solution.

In one aspect, the present invention provides compositions that, upon combination with water or other aqueous media, form an aqueous composition where the aqueous composition includes micelles. Thus, in one aspect, the present invention provides a composition comprising (a) a biocompatible copolymer selected from X—Y, X—Y—X and Y—X—Y having a hydrophilic block X and a hydrophobic block Y; (b) an additive selected from amino acid and oligopeptide; (c) a hydrophobic drug; and (d) water; where the composition includes micelles.

In another aspect, the present invention provides a composition that includes (a) a biocompatible copolymer selected from X—Y, X—Y—X and Y—X—Y having a hydrophilic block X, and a hydrophobic block Y; (b) an amino acid; (c) a hydrophobic drug; and (d) water; where the composition includes micelles.

In another aspect, the present invention provides compositions that, upon combination with water or other aqueous media, form an aqueous composition where the aqueous composition includes micelles. Thus, in one aspect, the present invention provides a composition comprising (a) a biocompatible diblock copolymer, X—Y, having a hydrophilic block X and a hydrophobic block Y; (b) an additive selected from amino acid and oligopeptide; (c) a hydrophobic drug; and (d) water; where the composition includes micelles.

In another aspect, the present invention provides a composition that includes (a) a biocompatible diblock copolymer, X—Y, having a hydrophilic block X, and a hydrophobic block Y; (b) an amino acid; (c) a hydrophobic drug; and (d) water; where the composition includes micelles. In one embodiment, the amino acid is selected from a naturally occurring amino acid. Examples of naturally occurring amino acid include, without limitation, alanine, arginine, asparagine, aspartic acid, citrulline, cysteine, cystine, glutamine, glycine, histidine, isoleucine, leucine, lysine, methionine, ornithine, phyenylalanine, proline, serine, threonine, tryptophen, tyrosine, valine, hydroxy proline, γ-carboxyglutamate, phenylglycine, or O-phosphoserine. In another embodiment, the amino acid is selected from non-naturally occurring amino acids. Exemplary non-naturally occurring amino acid include, without limitation, α-alanine, α-amino butyric acid, γ-amino butyric acid, γ-(aminophenyl) butyric acid, α-amino isobutyric acid, α-amino caproic acid, 7-amino heptanoic acid, β-aspartic acid, aminobenzoic acid, aminophenyl acetic acid, aminophenyl butyric acid, γ-glutamic acid, cysteine (ACM), ε-lysine, ε-lysine, (A-Fmoc), methionine sulfone, norleucine, norvaline, ornithine, d-ornithine, p-nitro-phenylalanine, hydroxy proline, 1,2,3,4,-tetrahydroisoquinoline-3-carboxylic acid and thioproline. When the additive is an oligopeptide, any one or more of these or other natural or non-natural amino acids may be present, in residue form, in the oligopeptide.

The micelle-containing compositions of the present invention may be readily prepared by adding aqueous media to the solid compostion of the copolymer, the hydrophobic drug and the amino acid or oligopeptide and then mixing the components together with some agitation at room temperature or an elevated temperature (e.g. 37° C. or 60° C.). The aqueous media will necessarily include water, and may be pure water, where pure water has less than 0.5 wt % dissolved solids. Pure water may be obtained by, e.g., distilling the water and/or subjecting the water to a deionization process and/or a reverse osmosis process. Both distillation, reverse osmosis and deionization of water is well known in the art, and many companies supply machines to efficiently purify water, see, e.g., Waters, Millipore, Mass. The aqueous media, in addition to water, may contain dissolved salts, e.g., NaCl or a buffer such as phosphate buffer, pH neutralizing agent such as a strong acid, e.g., HCl, or a strong base, e.g., NaOH or agents to modify the osmolality of the solution, e.g. dextrose. The aqueous media may also, or alternatively, contain one or more pharmaceutically acceptable carriers, as identified below. The composition of the present invention are particularly advantageous in that they form micelles at an enhanced rate, have an enhanced ability to incorporate drug(s); and/or have advantageous physical characteristics.

Thus, in one particular aspect of the present invention, a method is provided for forming a drug delivery vehicle. The method includes sequentially providing an aqueous or solid composition as describe herein; and adding aqueous media to the composition to form a micelle-containing composition. In addition to aqueous media, any pharmaceutically acceptable carriers may be used to constitute the therapeutic composition from the precursor composition. Such carriers are well known in the pharmaceutical art, and are described, for example, in Remingtons Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the composition. For example, sodium benzoate, sorbic acid and esters of p-hydroxybenzoic acid may be added as preservatives. Id. at 1449. In addition, antioxidants and suspending agents may be used. Id.

A micellar solution according to the present invention is preferably clear. A suitable test for the clarity of a solution is as follows. An aliquot of test sample is placed in a Quartz cuvette having a path length of 1 cm. The cuvette is placed in a UV spectrophotometer set to measure absorbance at 450 nm. Prior to analysis of the sample, the UV spectrophotometer is blanked using a normal saline control. An absorbance value of not greater than 0.15 AU for the test sample indicates the presence of a clear micellar solution. If the solution is not clear, or contains some insoluble hydrogel, the solution may be filtered.

In one aspect, the compositions of the present invention are sterile. Many pharmaceuticals are manufactured to be sterile and this criterion is defined by the USP XXII <1211>. The term “USP” refers to U.S. Pharmacopeia (see www.usp.org, Rockville, Md.). Sterilization in this embodiment may be accomplished by a number of means accepted in the industry and listed in the USP XXII <1211>, including gas sterilization, ionizing radiation or filtration. Sterilization may be maintained by what is termed asceptic processing, defined also in USP XXII <1211>. Acceptable gases used for gas sterilization include ethylene oxide. Acceptable radiation types used for ionizing radiation methods include gamma, for instance from a cobalt 60 source and electron beam. A typical dose of gamma radiation is 2.5 MRad. Filtration may be accomplished using a filter with suitable pore size, for example 0.22 μm and of a suitable material, for instance Teflon.

In another aspect, the compositions of the present invention are contained in a container that allows them to be used for their intended purpose, i.e., as a pharmaceutical composition. Properties of the container that are important are a volume of empty space to allow for the addition of a constitution medium, such as water or other aqueous medium, e.g., saline, acceptable light transmission characteristics in order to prevent light energy from damaging the composition in the container (refer to USP XXII <661>), an acceptable limit of extractables within the container material (refer to USP XXII), an acceptable barrier capacity for moisture (refer to USP XXII <671>) or oxygen. In the case of oxygen penetration, this may be controlled by including in the container, a positive pressure of an inert gas, such as high purity nitrogen, or a noble gas, such as argon.

Typical materials used to make containers for pharmaceuticals include USP Type I through III and Type NP glass (refer to USP XXII <661>), polyethylene, Teflon, silicone, and gray-butyl rubber. For parenterals, USP Types I to III glass and polyethylene are preferred.

In one aspect, the compositions of the present invention include one or more preservatives or bacteriostatic agents, present in an effective amount to preserve the composition and/or inhibit bacterial growth in the composition, for example, bismuth tribromophenate, methyl hydroxybenzoate, bacitracin, ethyl hydroxybenzoate, propyl hydroxybenzoate, erythromycin, chlorocresol, benzalkonium chlorides, and the like. Examples of the preservative include paraoxybenzoic acid esters, chlorobutanol, benzylalcohol, phenethyl alcohol, dehydroacetic acid, sorbic acid, etc. In one aspect, the compositions of the present invention include one or more bactericidal (also known as bacteriacidal) agents. In one aspect, the compositions of the present invention include one or more antioxidants, present in an effective amount. Examples of the antioxidant include sulfites and ascorbic acid. In one aspect, the compositions of the present invention include one or more coloring agents, also referred to as dyestuffs, which will be present in an effective amount to impart observable coloration to the composition. Examples of coloring agents include dyes suitable for food such as those known as F. D. & C. dyes and natural coloring agents such as grape skin extract, beet red powder, beta carotene, annato, carmine, turmeric, paprika, and so forth.

The present invention also provides a process of lyophilization, including lyophilization of the micelle-containing composition described above to create a lyophilized powder. In a preferred embodiment, the process further includes reconstitution of the lyophilized powder with water or other aqueous media, such as benzyl alcohol-containing bacteriostatic water for injection, to create a reconstituted solution (Bacteriostatic Water for Injection, Abbott Laboratories, Abbott Park, Ill.).

In another aspect, the present invention provides a method of treating a disease in a mammal including the administration of an effective amount of a micelle-containing composition as described above, or a precursor thereof that will form micelles in the mammal, to the mammal. In another aspect, the present invention provides a method of preventing disease in a mammal. The method includes the administration of an effective amount of a micelle-containing composition as described above, or a precursor thereof that will form micelles in the mammal, to the mammal. Examples of diseases are inflammatory conditions, surgical adhesions, osteoarthritis, neurological disorders, cancer, and benign hyperproliferative diseases. Thus, the disease may be arthritis, and/or the disease may be multiple sclerosis, and/or the disease may be Alzheimer's disease, and/or the disease may be psoriasis, and/or the disease may be cancer, and/or the disease may be stenosis or restenosis, and/or the disease may be benign hyperplasia, for example, benign hyperplasia induced by a foreign body, and/or the disease may be cardiovascular disease, and/or the disease may be Inflammatory Bowel Disease.

In one aspect, the composition is administered by a route selected from intravenous, intraarticular, intracutaneous, interstitial, subcutaneous, intramuscular injection, insertion into the rectum, oral, or implant into the body. The compositions of the invention may be administered in a solid form or in a liquid form. For example, the composition may be administered by intravenous delivery of an aqueous micelle solution. Alternatively, the composition may be administered by implanting a composition in the body, where the composition is a solid that releases the drug to the body over a period of time (e.g. hour to days).

In these therapeutic or prophylactic methods, a preferred hydrophobic drug being delivered by the method is paclitaxel or an analogue or derivative, thereof as described above.

In order to administer a composition of the present invention to a subject, in one method an aliquot of a micellar solution according to the present invention may be withdrawn using a syringe equipped with about a ca. 19 gauge needle. The aliquot is injected into an intravenous infusion bag and an additional quantity of 0.9% w/w saline solution is added to the infusion bag to yield a volume of 120 ml. The subject is then administered a therapeutically effective amount of the hydrophobic drug, from the intravenous infusion bag.

In general, the “therapeutically effective amount” of a hydrophobic drug according to the present invention will depend on the route of administration, the type of mammal being treated, and the physical characteristics of the specific mammal under consideration. These factors and their relationship to determining this amount are well known to skilled practitioners in the medical arts. This amount and the method of administration can be tailored to achieve optimal efficacy but will depend on such factors as weight, diet, concurrent medication and other factors which those skilled in the medical arts will recognize.

In one embodiment, the total amount of hydrophobic drug delivered per single dose administration is less than 5 grams. In another embodiment, the total amount of hydrophobic drug delivered per single dose administration is less than 1 gram. In yet another embodiment, the total amount of hydrophobic drug delivered per single dose administration is less than 0.5 gram.

The compositions of the present invention can be delivered a single administration or multiple administrations. The frequency of the multiple administrations can range from hours (e.g. hourly, every 2 hours, every 4 hours, every eight hours, every 12 hours or combinations thereof) to days (e.g. daily, every 2 days or every three days or combinations thereof) to weeks (e.g. weekly or biweekly and combinations thereof) to months (e.g. monthly, every 2 months, every 3 months, every 6 months or combinations thereof). The number of administrations delivered to the patient is dependant of the specific indication being treated and can range from a single administration to on-going administrations for the duration of the disease or condition or the life-time of the patient.

The following examples are offered by way of illustration, and not by way of limitation. In the Examples, paclitaxel was obtained from Hauser Chemical (Boulder, Colo.); DL-lactide was obtained from Purac America (Lincolnshire, Ill.); and MePEG was obtained from Sigma (St. Louis, Mo.). Other materials were obtained from the suppliers listed in Table 3.

TABLE 3
Summary of Materials Used

| Material | Supplier | Lot Number |
|---|---|---|
| Diblock Polymer | Angiotech | LN D00074 |
| Paclitaxel | Hauser | 1492-11199-a |
| | | LN 1492-13599-A |
| Tetrahydrofuran | Aldrich, | KU 01049KU |
| | Caledon | 35154 |
| 0.9% NaCl Irrigation | B. Braun | J0N159 |
| Na₂HPO₄.7H₂O | Sigma | 12H64481 |
| NaH₂PO₄.H₂O | Fisher | 968020 |
| L-Alanine | Aldrich | DO 1601100 |
| L-Arginine | Aldrich | LO 09314KO |
| L-Asparagine | Aldrich | AO 22022AO |
| L-Aspartic acid | Aldrich | AO 08812LI |
| L-Cysteine | Sigma | 125H0288 |
| L-Glutamine | Aldrich | CO 02701CO |
| L-Glutamic acid | Aldrich | AO 03818LR |
| Glycine | Sigma | 36H00791 |
| L-Histidine | Aldrich | JI 04821JR |
| L-Isoleucine | Aldrich | EI 12728HU |
| L-Leucine | Aldrich | CO 12320CI |
| L-Lysine | Aldrich | KI 08325JI |
| L-Methionine | Aldrich | PI 05512MI |
| L-Phenylalanine | Aldrich | TI 03323LU |
| L-Proline | Aldrich | JO 17324EO |
| D-Serine | Aldrich | LO 10414KO |
| L-Serine | Aldrich | KI 02528JI |
| L-Threonine | Aldrich | EU 13421CO |
| L-Tryptophane | Aldrich | HO 30419CO |
| L-Tyrosine | Aldrich | MI 07006EU |
| D-Valine | Aldrich | CO 0951KU |
| L-Valine | Aldrich | CO 06024JR |
| MePEG 2000 | Fluka | V99131 |

The molecular weight and molecular weight distribution of a polymer or block copolymer may be determined using gel permeation chromatography (GPC) according to techniques well known in the art, where many manufacturers sell instruments for this purpose, see, e.g., Waters, Milford, Mass. Typically, the retention time(s) for a sample polymer, as determined by GPC, are compared to the retention time(s) of monodisperse polystyrene standards (which are commercially available from many suppliers, e.g., Aldrich Chemical, Waters, and Showa Denko, Japan are three representative suppliers), and this comparison provides a molecular weight measurement for the sample polymer. The average diameter of a micelle particle, and the size distribution of the particles, may be determined by light scattering techniques well known in the art. See, e.g., K. Holmberg, ed., Handbook of Applied Surface and Colloid Chemistry, John Wiley & Sons, 2001. For instance, particle size may be determined by subjecting a micellar solution to dynamic light scattering (DLS) spectrometry, where a suitable spectrometer is available from many commercial suppliers, e.g., Lexel Laser Inc. Freemont, Calif. and Brookhaven Instruments Co., Holtsville, N.Y. The spectrometer can be set at a wavelength of about 500 nm and a temperature of about 25° C. in making the measurements. A suitable detector for the scattered light is a photomultiplier, where photomultipliers are likewise available from many commercial suppliers, e.g., Brookhaven Instruments Co., Holtsville, N.Y.

### EXAMPLES

### Starting Material Preparations and General Procedures

A. Preparation of 60:40 MePEG:Poly(DL-lactide) Diblock Copolymer

A 60:40 MePEG:poly(DL-lactide) diblock copolymer was prepared by combining 60 g of DL-lactide and 40 g of MePEG (MW=2,000 g/mol) in a round bottom glass flask containing a TEFLON™-coated stir bar. The mixture was heated to 140° C. with stirring in a temperature controlled mineral oil bath until the components melted to form a homogeneous liquid. 0.1 g of stannous 2-ethyl hexanoate was added to the molten mixture and the reaction was continued for 6 hours at 140° C. with continuous stirring. The reaction was terminated by cooling the product to ambient temperature. The product, 60:40 MePEG:poly(DL-lactide) diblock copolymer was stored in sealed containers at 2-8° C. until use.

B. Precipitation of 60:40 MePEG:Poly(DL-lactide) Diblock Copolymer from Isopropanol

A 60:40 MePEG:poly(DL-lactide) diblock copolymer was prepared according to the method in Example 1. The copolymer (47 g) was dissolved in isopropanol to make a 5% w/v solution. The mixture was heated to 50° C. for 2 hours with shaking every 30 minutes. The result was a clear solution. The solution was cooled to 2° C. over a 16 hour period in order to precipitate the copolymer. The mixture was centrifuged for 20 minutes at 3000 rpm to pelletize the copolymer and the supernatant was removed and replaced with fresh isopropanol. The heating (to dissolve) and cooling (to precipitate) cycle was repeated twice more. After the final precipitation step, the copolymer pellet was transferred to a vacuum oven cooled to −10° C. and dried for 5 hours. The oven was heated to ambient temperature and the vacuum maintained for a further 10 hours. The result was copolymer recovered as a white powder.

At various times in drying, the copolymer was analyzed by head space gas chromatography to measure the amount of isopropanol remaining in the matrix. The analysis was performed using a Supelcowax-10 GC column (30 m×530 μm×1.00 μm nominal), an injection port temperature of 140° C., detector temperature of 260° C. and column temperature of 50° C. for 9 minutes after injection, increasing thereafter to 100° C. at 12° C./min. Typical values of isopropanol remaining were as follows:

| | Lower | Upper |
|---|---|---|
| Drying time (h) | Range (%/w/w) | Range (% w/w) |
| 42 | 0.05 | 1.32 |
| 104 | 0.02 | 0.04 |

Higher values were observed in a batch made which resulted in a coarser powdered product. In this batch the amounts of isopropanol remaining were as follows:

| | Lower | Upper |
|---|---|---|
| Drying time (h) | Range (%/w/w) | Range (% w/w) |
| 42 | 5.87 | 6.74 |
| 104 | 1.57 | 6.03 |

In order to test an alternate drying system, prior to drying the copolymer by reduced pressure, small aliquots of approximately 3 grams were removed and placed at 50-55° C. with heated air forced over the samples. At intervals of 24 and 48 hours, these small samples were removed at analyzed in the same manner by GC. In these samples, no isopropanol was detected. The limit of detection of the assay is less than 0.01% w/w. An alternative GC method may be found for isopropanol in USP XXIV <467>.

Accordingly, in various aspects the present invention provides diblock copolymer having isopropanol contamination of less than 10% w/w, less than 5% w/w/, less than 1% w/w, less than 0.1% w/w/, less than 0.01% w/w. A preferred diblock copolymer having minimual isopropanol contamination is a polyester-polyether diblock coplymer, and a further preferred diblock copolymer is a poly(ethylene glycol)-poly(lactide) copolymer. Drying techniques as described herein may also be used to remove other solvent contamination, so as to provide diblock copolymer having solvent contamination of less than 10% w/w, less than 5% w/w/, less than 1% w/w, less than 0.1% w/w/, and less than 0.01% w/w, where an exemplary contaminating solvents besides isopropanol is tetrahydrofuran.

C. Buffer Preparation

Weigh out 5.5077 g of Na₂HPO₄.7H₂O, and 0.3545 g of NaH₂PO₄.H₂O into a 500 mL Erlenmeyer flask. 500 mL of 0.9% NaCl solution is then added into the flask. The solution is stirred until all the material dissolved and a homogeneous mixture is produced.

D. Freeze Drying Procedure

Before putting a sample into the freeze-dryer, the freeze-dryer has to be cooled to −34° C. for several hours to ensure the shelves have reached a stable temperature. Remove the cap of the vials, and put them into the cooling chamber. Since different shelves may have different cooling profiles, some care in tracking where samples are placed should be taken. Maintain the shelf temperature at −34° C. for at least an hour before starting the vacuum. While under vacuum, the samples were maintained at −34° C. for an additional two hours, after which the temperature was slowly increased to −20° C. at a rate of 1° C./min. This low temperature was maintained for 24 hours. The temperature was then ramped to −5° C. at 1° C./min. The shelf temperature is maintained at −5° C. for at least 24 hours before the samples were removed.

### EXAMPLE 1

### Effect of Amino Acids on Resuspension of Freeze Dried Micellar Paclitaxel

A set of 10 samples was prepared by weighing 990 mg of 60:40 MePEG:Poly(DL-lactide) diblock copolymer into a 50 mL Erlenmeyer flask. The copolymer was dissolved in 20 mL tetrahydrofuran. 110 mg of paclitaxel was then added to the polymer solution. Aliquots of 2 mL each were placed into 10 scintillation vials, with each vial containing 2 mL copolymer/paclitaxel solution. Accordingly, each sample contained 99 mg of copolymer, 11 mg of paclitaxel and 2 mL of tetrahydrofuran prior to drying. The vials, with caps removed, were placed in a forced-air oven at 50° C. for about 1 hr 15 minutes. The dried samples vials were transferred to a vacuum oven and dried in vacuo overnight to remove any residual solvent.

After overnight drying under vacuum, the dried sample vials were removed from the vacuum oven. To each vial was added 5 mL of buffer solution, prepared as described in Preparation C. The vials were agitated until all the copolymer/paclitaxel dissolved. A desired amount of amino acid was added to each solution. The amount of amino acid added depended on the specific experiment.

The amounts of L-leucine and L-isoleucine added in the specific experiments were not completely soluble in 5 mL of buffer. Upon incomplete dissolution, additional buffer was added to dissolve these amino acids when the amount of amino acid added exceeded 55 mg per vial.

The samples were then dried by freeze-drying, as described in General Procedure D. Most of the samples containing D/L-serine were partially or completely collapsed after the freeze-dried process. Two third of the samples containing 55 mg L-alanine collapsed after freeze-drying, and more than 90% of the samples containing 55 mg glycine had potential to collapse or were partially collapsed. These data suggested that the cakes with D/L-serine, L-alanine, glycine and were not stable under the freeze-dried conditions described above. For these amino acid samples, the freeze drying program could be modified to prevent cake collapse. No collapsed cakes were observed among the control samples and the samples containing other amino acids.

The freeze-dried cakes were dissolved in 5 mL of 0.9% NaCl solution and the dissolution times were recorded. When no amino acid was added, the sample served as a control. Control samples were repeatedly tested for dissolution time, as shown in Table 4, in order to obtain a standard deviation for the time needed to resuspend amino-acid free samples.

TABLE 4
Average Dissolution time of control samples

| Control | Dissolution |
|---|---|
| Sample # | Time(s) |
| 1 | 40 |
| 2 | 43 |
| 3 | 75 |
| 4 | 120 |
| 5 | 77 |
| 6 | 126 |
| 7 | 123 |
| 8 | 118 |
| 9 | 140 |
| 10 | 67 |
| 11 | 71 |
| 12 | 75 |
| 13 | 66 |
| 14 | 99 |
| 15 | 73 |
| 16 | 61 |
| 17 | 54 |
| 18 | 117 |
| 19 | 69 |
| 20 | 65 |
| 21 | 72 |
| 22 | 99 |
| 23 | 97 |
| 24 | 73 |
| 25 | 66 |
| 26 | 60 |
| 27 | 70 |
| 28 | 53 |
| 29 | 60 |
| 30 | 63 |
| 31 | 55 |
| 32 | 60 |
| 33 | 79 |
| 34 | 50 |
| 35 | 45 |
| 36 | 55 |
| Average | 76 |
| STD | 26 |

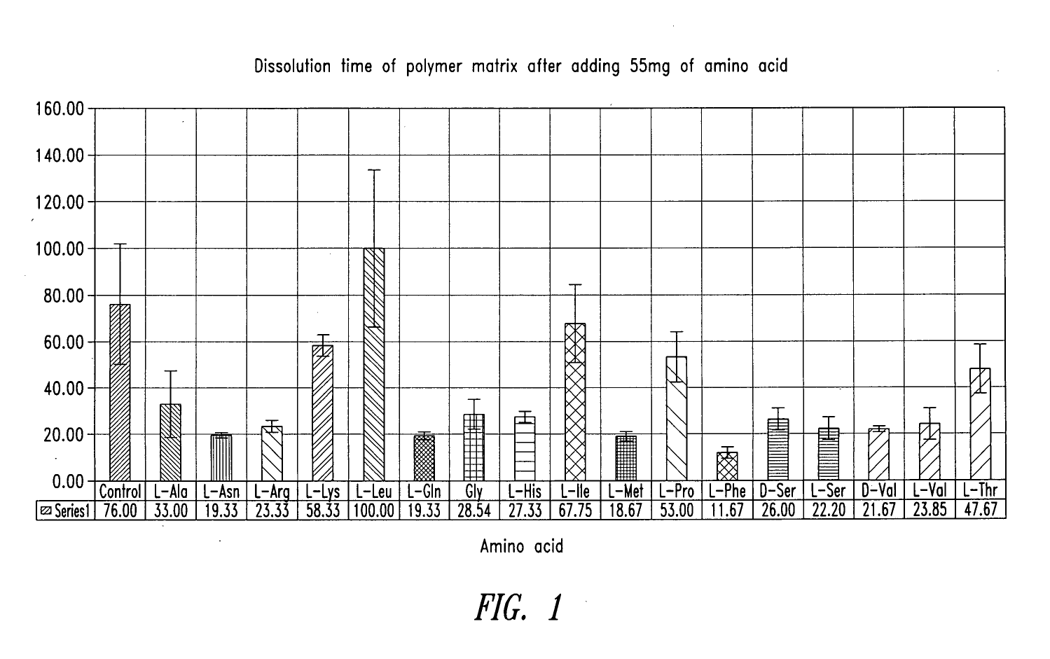

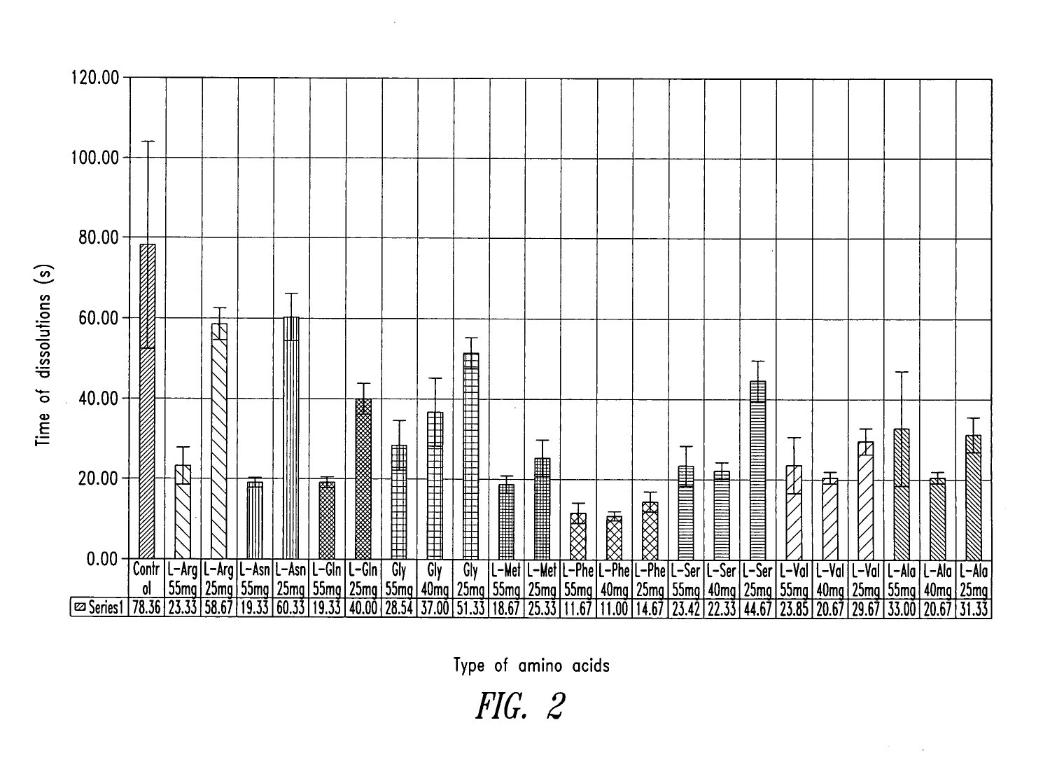

The results from these experiments are shown in FIGS. 1 and 2, with the actual data shown in Tables 5A and 5B. Although most of the D/L-serine containing samples were collapsed, there was still a large decrease in the time required for dissolution compared with the control samples, as shown in FIG. 1. The result indicated that the collapsed cakes and the freeze-drying process did not have profound impact on the dissolution time under these experimental conditions.

Effect of Amino Acid Stereochemistry on Dissolution Time.

D-valine and D-serine were tested in order to determine whether the enantiomeric properties of the amino acids had any effect on micelle dissolution. The dissolution time of the D-serine was 26.00±5.20 seconds and the L-serine was 23.42±5.12 seconds. The D-valine was 21.67±1.53 seconds and the L-valine was 23.85+6.89 seconds. These results would indicate that there were no significant differences in the dissolution times between the amino acid enantiomers under these experimental conditions.

Effect of Amino Acid Composition of Dissolution Rates

The comparison of the dissolution times between 55 mg of various amino acids is summarized in FIG. 1. L-phenylalanine had the lowest average dissolution time of 13.00±3.00 seconds. Compared with the control, in which no amino acid was added and which had a dissolution time of 78.36±25.65 seconds, the L-phenylalanine decreased the dissolution time by about 83.4%. Although possessing similar structures, samples with L-serine dissolved within 22 seconds, whereas samples with L-threonine dissolved within 47 seconds. Although the L-leucine containing samples showed the highest dissolution time at 100.00±33.67 seconds, the standard deviation was also high.

Effect of Amino Acid Concentration on Dissolution Time

It was observed that decreasing the amount of amino acids from 55 mg to 40 mg and 25 mg increased the dissolution time of the diblock polymer and paclitaxel matrix as compared to the control, as shown in FIG. 2. A general trend was observed. The dissolution time of the sample decreased as the amount of amino acid increased. The dissolution time was the largest for the samples containing 25 mg of amino acid, with the exception of L-alanine. The samples with 55 mg and 25 mg of L-alanine gave dissolution times with nearly identical results at about 33.00±14.22 and 31.33±4.51 seconds respectively. Sample with 40 mg L-alanine showed the shortest dissolution time at 20.67±1.53 seconds. However, the standard deviation of the samples containing 55 mg of L-alanine was large, which indicated there could be some experimental errors. The samples containing L-valine had dissolution time all within 30 seconds, and the differences in the results were not significant, considering their standard deviations. The samples containing 40 mg and 55 mg of L-serine gave an average dissolution time of 22.33 and 22.20 seconds. The 25 mg L-serine samples showed an average dissolution time of two fold larger than that of the 40 mg and 55 mg. The dissolution times of different amounts of glycine were also shown.

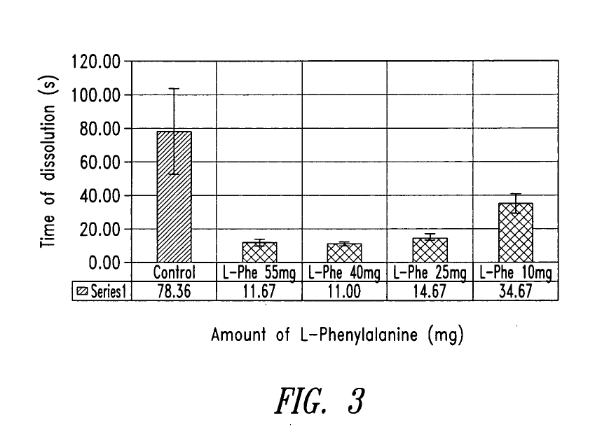

At each tested concentration, the L-phenylalanine-containing samples had the lowest dissolution time among all the other amino acids, which was the same conclusion obtained in FIG. 1. Since the dissolution time of the sample with L-phenylalanine was the shortest, further experiments were conducted to investigate the effect of amino acid concentration on the dissolution times. Samples with 10 mg, 25 mg, 40 mg and 55 mg of L-phenylalanine were tested and compared, and the results were depicted in FIG. 3. The differences between 25 mg, 40 mg and 55 mg were not significant; they dissolved within 11-14 seconds. Samples with only 10 mg of L-phenylalanine, however, had an increased dissolution time of 34 seconds. This dissolution time was however greatly shorter that the average dissolution time of 76 sec for the control samples.

TABLE 5A
Summary of dissolution time of polymer matrix
after adding 55 mg of amino acids

| Amino | L- | L- | | | | | | |
|---|---|---|---|---|---|---|---|---|
| acids | Ala | Lys | L-Asn | L-Leu | L-Ile | L-Pro | L-Gln | Gly |
| 1 | 56 | 60 | 20 | 150 | 90 | 45 | 20 | 35 |
| 2 | 54 | 53 | 20 | 80 | 66 | 66 | 18 | 40 |
| 3 | 42 | 62 | 18 | 90 | 65 | 48 | 20 | 29 |
| 4 | 22 | | | 80 | 50 | | | 24 |
| 5 | 21 | | | | | | | 27 |
| 6 | 24 | | | | | | | 25 |
| 7 | 33 | | | | | | | 23 |
| 8 | 22 | | | | | | | 42 |
| 9 | 23 | | | | | | | 30 |
| 10 | | | | | | | | 21 |
| 11 | | | | | | | | 27 |
| 12 | | | | | | | | 25 |
| 13 | | | | | | | | 23 |
| Avg | 33 | 58 | 19 | 100 | 68 | 53 | 19 | 29 |
| STD | 14 | 4.7 | 1.2 | 34 | 16 | 11 | 1.2 | 6.6 |

TABLE 5B
Summary of dissolution time of polymer matrix
after adding 55 mg of amino acids

| Amino | L- | L- | L- | D- | L- | D- | L- | L- | L- |
|---|---|---|---|---|---|---|---|---|---|
| acids | His | Met | Phe | Ser | Ser | Val | Val | Arg | Thr |
| 1 | 27 | 20 | 10 | 32 | 26 | 22 | 26 | 26 | 44 |
| 2 | 25 | 16 | 13 | 23 | 31 | 23 | 18 | 21 | 59 |
| 3 | 30 | 20 | 16 | 23 | 23 | 20 | 23 | 23 | 61 |
| 4 | | | 11 | | 17 | | 45 | | 47 |
| 5 | | | 9 | | 13 | | 20 | | 34 |
| 6 | | | 11 | | 19 | | 25 | | 41 |
| 7 | | | | | 28 | | 23 | | |
| 8 | | | | | 27 | | 20 | | |
| 9 | | | | | 25 | | 18 | | |
| 10 | | | | | 27 | | 20 | | |
| 11 | | | | | 24 | | 25 | | |
| 12 | | | | | 21 | | 23 | | |
| 13 | | | | | 20 | | 24 | | |
| 14 | | | | | 16 | | | | |
| 15 | | | | | 16 | | | | |
| Avg | 27 | 19 | 12 | 26 | 22 | 22 | 24 | 23 | 48 |
| STD | 2.5 | 2.3 | 2.5 | 5.2 | 5.3 | 1.5 | 6.9 | 2.5 | 10 |

### EXAMPLE 2

### Effect of Amino Acids in Combination with MePEG on Resuspension of Freeze Dried Micellar Paclitaxel

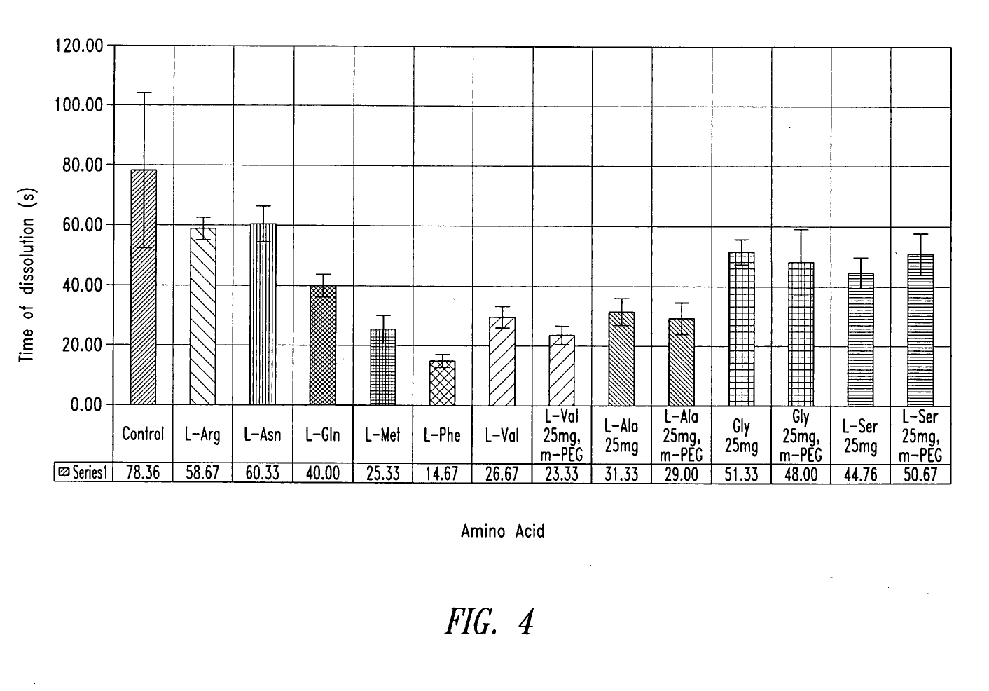

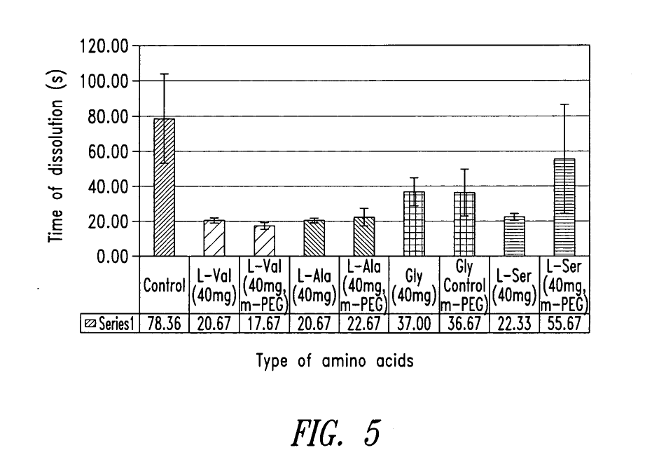

Samples were prepared as in Example 1, with the exception that 10 mg of MePEG 2000 per vial was added to some of the polymer preparations before dispersing into the scintillation vials. The results of resuspension upon the addition of buffer is shown in FIGS. 4 and 5, with the actual data presented in Tables 6 and 7.

In the cases of L-valine, L-alanine and glycine, the effect of MePEG 2000 on resuspension was minimal. However, in the case of L-serine, the addition of MePEG 2000 increased the dissolution time by about two fold when the L-serine level was 40 mg (FIG. 5). When the L-serine level was reduced to 25 mg, the effect of MePEG was not so dramatic (FIG. 4). The data corresponding to these Figures is provided in Tables 6 and 7.

TABLE 6
Summary of dissolution time of polymer matrix
after adding 25 mg of amino acids

| Amino | | | | | | | L-Val | | L-Ala | | Gly | | L-Ser |
|---|---|---|---|---|---|---|---|---|---|---|---|---|---|
| acids | L-Arg | L-Asn | L-Gln | L-Met | L-Phe | L-Val | m-PEG | L-Ala | m-PEG | Gly | m-PEG | L-Ser | m-PEG |
| 1 | 55 | 67 | 44 | 20 | 16 | 30 | 26 | 27 | 34 | 56 | 54 | 40 | |
| 2 | 58 | 58 | 36 | 29 | 18 | 36 | 24 | 31 | 23 | 48 | 55 | 44 | |
| 3 | 63 | 56 | 40 | 27 | 16 | 28 | 20 | 36 | 30 | 50 | 35 | 50 | |
| 4 | | | | | 14 | 30 | | | | | | | |
| 5 | | | | | 12 | 28 | | | | | | | |
| 6 | | | | | 12 | 26 | | | | | | | |
| Avg | 59 | 60 | 40 | 25 | 15 | 30 | 23 | 31 | 29 | 51 | 48 | 45 | |
| STD | 4.0 | 5.9 | 4.0 | 4.7 | 2.4 | 3.4 | 3.1 | 4.5 | 5.6 | 4.2 | 11 | 5.0 | |

TABLE 7
Summary of dissolution time of polymer matrix
after adding different amount of amino acids

| | | | | L-Val | | L-Ala | | Gly | | L-Ser |
|---|---|---|---|---|---|---|---|---|---|---|
| Amino | L-Phe | L-Phe | L-Val | 40 mg | L-Ala | 40 mg | Gly | 40 mg | L-Ser | 40 mg |
| acids | 10 mg | 40 mg | 40 mg | m-PEG | 40 mg | m-PEG | 40 mg | m-PEG | 40 mg | m-PEG |
| 1 | 36 | 12 | 22 | 16 | 22 | 22 | 30 | 28 | 20 | 68 |
| 2 | 28 | 10 | 19 | 20 | 19 | 18 | 46 | 30 | 24 | 22 |
| 3 | 40 | 11 | 21 | 17 | 21 | 28 | 35 | 52 | 23 | 80 |
| 4 | | | | | | | | | | |
| 5 | | | | | | | | | | |
| 6 | | | | | | | | | | |
| Avg | 35 | 11 | 21 | 18 | 21 | 23 | 37 | 37 | 22 | 57 |
| STD | 6.1 | 1.0 | 1.5 | 2.1 | 1.5 | 5.0 | 8.2 | 13 | 2.1 | 31 |

### EXAMPLE 3

### Determination of Completeness of a Micellar Solution

To determine whether a residue has been completely reconstituted to form micelles, an aqueous composition is analyzed for its UV absorbance in a Quartz cuvette having a path length of 1 cm using a UV spectrophotometer set to measure absorbance at 450 nm. Prior to analysis the UV spectrophotometer is blanked using a normal saline control. An absorbance value of not greater than 0.15 AU indicates the presence of a clear micellar solution.

All of the above U.S. patents, U.S. patent application publications, U.S. patent applications, foreign patents, foreign patent applications and non-patent publications referred to in this specification and/or listed in the Application Data Sheet, including but not limited to U.S. application Ser. No. 10/251,659, are incorporated herein by reference, in their entirety.

From the foregoing, it will be appreciated that, although specific embodiments of the invention have been described herein for purposes of illustration, various modifications may be made without deviating from the spirit and scope of the invention.

1. A composition comprising:
(a) a micelle-forming biocompatible diblock copolymer (X—Y) having a hydrophilic block X comprising residues of monomer x, and a hydrophobic block Y comprising residues of monomer y;
(b) amino acid; and
(c) a hydrophobic drug;
with the proviso that the composition forms a micellar solution in water.

2. A composition comprising:
(a) a micelle-forming biocompatible diblock copolymer (X—Y) having a hydrophilic block X comprising residues of monomer x, and a hydrophobic block Y comprising residues of monomer y;
(b) oligopeptide; and
(c) a hydrophobic drug;
with the proviso that the composition forms a micellar solution in water.

3. A composition comprising:
(a) a biocompatible diblock copolymer (X—Y) having a block X comprising residues of monomer x, and a block Y comprising residues of monomer y, the block X being more hydrophilic than the block Y;
(b) an additive selected from amino acid and oligopeptide; and
(c) a hydrophobic drug;
with the proviso that the composition forms a micellar solution in aqueous media.

4. A composition comprising:
(a) a micelle-forming biocompatible block copolymer having a Y—X—Y or X—Y—X structure, wherein the copolymer has a hydrophilic block X comprising residues of monomer x, and a hydrophobic block Y comprising residues of monomer y;
(b) amino acid; and
(c) a hydrophobic drug;
with the proviso that the composition forms a micellar solution in water.

5. A composition comprising:
(a) a micelle-forming biocompatible block copolymer having a Y—X—Y or X—Y—X structure, wherein the copolymer has a hydrophilic block X comprising residues of monomer x, and a hydrophobic block Y comprising residues of monomer y;
(b) oligopeptide; and
(c) a hydrophobic drug;
with the proviso that the composition forms a micellar solution in water.

6. A composition comprising:
(a) a biocompatible block copolymer having a Y—X—Y or X—Y—X structure, wherein the copolymer has a block X comprising residues of monomer x, and a block Y comprising residues of monomer y, the block X being more hydrophilic than the block Y;
(b) an additive selected from amino acid and oligopeptide; and
(c) a hydrophobic drug;
with the proviso that the composition forms a micellar solution in aqueous media.

7. The composition of claim 1 wherein the block X comprises residues of one or more monomers selected from (meth)acrylic acid, styrene sulfonate, 2-acrylamido-2-methyl propane sulfonic acid, acrylamide, vinylpyrrolidone, saccharide, and amino acid.

8. The composition of claim 1 wherein the block X comprises residues of alkylene oxide.

9. The composition of claim 1 wherein block X comprises poly(alkylene oxide).

10. The composition of claim 9 wherein the poly(alkylene oxide) is selected from poly(ethylene oxide) and terminal C₁-C₆ alkyl ethers of poly(ethylene oxide).

11. The composition of claim 10 wherein the terminal C₁-C₆ alkyl ether of the polyethylene oxide is methoxy polyethylene oxide.

12. The composition of claim 9 wherein the poly(alkylene oxide) is poly(ethylene oxide).

13. The composition of claim 1 wherein the block Y comprises residues of monomers selected from methacrylic acid, esters of methacrylic acid, esters of acrylic acid, styrene, and vinyl acetate.

14. The composition of claim 1 wherein the block Y comprises residues of monomers selected from lactic acid and reactive equivalents thereof, glycolic acid and reactive equivalents thereof, caprylic acid and reactive equivalents thereof, trimethylene carbonate, 1,4-dioxane-2-one, and 1,5-dioxepan-2-one.

15. The composition of claim 14 wherein the block Y is poly-DL-lactide-co-glycolide.

16. The composition of claim 14 wherein the block Y is poly-DL-lactide.

17. The composition of claim 1 wherein block X comprises residues of monomers selected from alkylene oxide, acrylic acid, vinyl pyrrolidone, saccharide, and amino acid, and block Y comprises residues of monomers selected from lactide or reactive equivalents thereof, glycolide or reactive equivalents thereof, caprolactone or reactive equivalents thereof, hydrophobic amino acid, carbonate, and vinyl acetate.

18. The composition of claim 17 wherein block X comprises residues of alkylene oxide, and block Y comprises residues of monomers selected from lactide or reactive equivalents thereof, glycolide or reactive equivalents thereof, caprolactone or reactive equivalents thereof, trimethylene carbonate, 1,4-dioxane-2-one, and 1,5-dioxepan-2one.

19. The composition of claim 18 wherein block X comprises residues of alkylene oxide and block Y comprises residues of monomers selected from lactide or reactive equivalents thereof and glycolide or reactive equivalents of glycolide.

20. The composition of claim 17 wherein the alkylene oxide is ethylene oxide.

21. The composition of claim 18 wherein the block Y comprises residues of lactide.

22. The composition of claim 17 wherein block X comprises methoxy polyethylene oxide.

23. The composition of claim 22 wherein block Y comprises poly(DL-lactide).

24. The composition of claim 1 wherein 100 parts of diblock copolymer comprise 40-90 parts hydrophilic polymer X and 60-10 parts hydrophobic polymer Y.

25. The composition of claim 24 wherein 100 parts of diblock copolymer comprise 40-80 parts hydrophilic polymer X and 60-20 parts hydrophobic polymer Y.

26. The composition of claim 24 wherein 100 parts of diblock copolymer comprise 50-70 parts hydrophilic polymer X and 50-30 parts hydrophobic polymer Y.

27. The composition of claim 24 wherein 100 parts of diblock copolymer comprise about 60 parts hydrophilic polymer X and about 40 parts hydrophobic polymer Y.

28. The composition of claim 1 wherein the diblock copolymer has a number average molecular weight of about 1,000 to about 10,000 g/mol.

29. The composition of claim 28 wherein the diblock copolymer has a number average molecular weight of about 2,000 to about 5,000 g/mol.

30. The composition of claim 28 wherein the diblock copolymer has a number average molecular weight of about 2,500 to about 3,500 g/mol.

31. A composition of claim 1 wherein the amino acid has a water-solubility of greater than about 2.5 g per 100 g water at 25° C.

32. A composition of claim 31 where the amino acid is selected from the L and D isomers of alanine, arginine, asparagines, cysteine, glutamine, glycine, histidine, isoleucine, lysine, methionine, phenylalanine, proline, threonine, and valine.

33. A composition of claim 2 wherein the oligopeptide has a water-solubility of greater than about 2.5 g per 100 g water at 25° C.

34. The composition of claim 1 wherein the amino acid is a naturally occurring amino acid.

35. The composition of claim 34 wherein the amino acid is selected from L and D isomers of alanine, arginine, asparagine, aspartic acid, cysteine, glutamine, glutamic acid, glycine, histidine, isoleucine, leucine, lysine, methionine, phenylalanine, proline, serine, threonine, tryptophane, tyrosine, and valine.

36. The composition of claim 1 wherein the amino acid is a non-naturally occurring amino acid.

37. The composition of claim 36 wherein the non-naturally occurring amino acid is selected from the group consisting of β-alanine, α-amino butyric acid, γ-amino butyric acid, γ-(aminophenyl) butyric acid, α-amino isobutyric acid, ε-amino caproic acid, 7-amino heptanoic acid, β-aspartic acid, aminobenzoic acid, aminophenyl acetic acid, aminophenyl butyric acid, γ-glutamic acid, cysteine (ACM), ε-lysine, ε-lysine, (A-Fmoc), methionine sulfone, norleucine, norvaline, ornithine, d-ornithine, p-nitro-phenylalanine, hydroxy proline, 1,2,3,4,-tetrahydroisoquinoline-3-carboxylic acid and thioproline.

38. The composition of claim 1 further comprising MePEG.

39. The composition of claim 38 wherein the MePEG has a molecular weight of 200-750 g/mol.

40. The composition of claim 38 wherein the MePEG has a molecular weight of 550-2000 g/mol

41. The composition of claim 38 wherein the MePEG has a molecular weight of 750-5000 g/mol.

42. The composition of claim 38 wherein the MePEG has a molecular weight of 200-5000 g/mol.

43. The composition of claim 1 comprising about 1 to about 5 parts block copolymer per each 1 part additive, on a weight basis.

44. The composition of claim 1 wherein the hydrophobic drug is selected from the group consisting of chemotherapeutic, antibiotic, antimicrobial, antimicrotubule, anti-inflammatory, immunosuppressant and antiproliferative drugs.

45. The composition of claim 1 wherein the drug is selected from paclitaxel, paclitaxel derivatives and paclitaxel analogues.

46. The composition of claim 1 wherein the drug is paclitaxel.

47. The composition of claim 1 further comprising a buffering constituent.

48. The composition of claim 47 wherein the buffering constituent comprises a phosphate salt.

49. The composition of claim 1 comprising 10-90 parts diblock copolymer, 10-70 parts additive selected from amino acid and oligopeptide, 1-15 parts paclitaxel and 1-20 parts phosphate salt.

50. The composition of claim 1 comprising about 50-80 parts of diblock copolymer, about 10-40 parts additive selected from amino acid and oligopeptide, about 8 parts paclitaxel and about 18 parts phosphate salt, the parts in weight totaling 100.

51. The composition of claim 1 comprising about 55-75 parts diblock copolymer, about 15-35 parts additive selected from amino acid and oligopeptide, about 7 parts paclitaxel and about 11 parts phosphate salt, the parts in weight totaling 100.

52. The composition of claim 1 having less than 5% moisture content.

53. The composition of claim 52 having less than 0.5% moisture content.

54. The composition of claim 52 wherein the composition is sterile.

55. The compositions of 54 claim 1 wherein the composition is produced through lyophilization of a micellar solution.

56. The composition of claim 54 wherein the composition is packaged within a container that maintains the sterility of the composition.

57. The composition of claim 56 wherein the composition is packaged within packaging comprising a glass container with a sealed closure.

58. The composition of claim 56 wherein the composition is packaged within packaging comprising a plastic container with a sealed closure.

59. The composition of claims 56 wherein the packaging further comprises a sufficient volume of empty space to allow for the addition of water in a sufficient amount to produce a micelle-containing composition.

60. The composition of claims 56 wherein the packaging is substantially opaque to UV or visible light.

61. The composition of claims 56 wherein the packaging is substantially impervious to oxygen from air.

62. The composition of claim 1 further comprising a bacteriacidal or bacteriostatic compound.

63. The composition of claim 1 further comprising an antioxidant.

64. The composition of claim 1 further comprising a coloring agent.

65. A method of producing the composition according to claim 52 comprising treating the composition according to a sterilization process selected from sterile filtration, sterilization with ethylene oxide, and sterilization with ionizing radiation.

66. The composition of claim 1 further comprising water to form an aqueous composition, the aqueous composition comprising micelles.

67. A composition comprising:
(a) a biocompatible diblock copolymer (X—Y) having a hydrophilic block X and a hydrophobic block Y;
(b) amino acid,
(c) a hydrophobic drug; and
(d) water;
wherein the composition comprises micelles.

68. A composition comprising
(a) a biocompatible diblock copolymer (X—Y) having a hydrophilic block X, and a hydrophobic block Y;
(b) an oligopeptide;
(c) a hydrophobic drug; and
(d) water;
wherein the composition comprises micelles.

69. A composition comprising
(a) a biocompatible diblock copolymer (X—Y) having a hydrophilic block X, and a hydrophobic block Y;
(b) two different amino acids;
(c) a hydrophobic drug; and
(d) water;
wherein the composition comprises micelles.

70. A composition comprising
(a) a biocompatible block copolymer having a X—Y—X or Y—X—Y structure, wherein the copolymer has a hydrophilic block X and a hydrophobic block Y;
(b) amino acid,
(c) a hydrophobic drug; and
(d) water;
wherein the composition comprises micelles.

71. A composition comprising
(a) a biocompatible block copolymer having a X—Y—X or Y—X—Y structure, wherein the copolymer has a hydrophilic block X, and a hydrophobic block Y;
(b) an oligopeptide;
(c) a hydrophobic drug; and
(d) water;
wherein the composition comprises micelles.

72. A composition comprising
(a) a biocompatible block copolymer having a X—Y—X or Y—X—Y structure, wherein the copolymer has a hydrophilic block X, and a hydrophobic block Y;
(b) two different amino acids;
(c) a hydrophobic drug; and
(d) water;
wherein the composition comprises micelles.

73. A method for forming a drug delivery vehicle, comprising:
(a) providing the composition of claim 1; and
(b) adding water to the composition to form a micelle-containing composition.

74. A method of forming a composition of claim 1 comprising sequentially:
(a) combining the diblock copolymer, amino acid or oligopeptide additive and hydrophobic drug with an additional processing solvent; and
(b) removing the processing solvent by evaporation or distillation.

75. A method according to claim 78 wherein the processing solvent comprises an organic solvent selected from the group consisting of tetrahydrofuran, ethanol, acetonitrile, chloroform, and dichloromethane.

76. A method according to claim 74 further comprising adding water to the composition.

77. A method of treating a disease in a mammal comprising the administration of an effective amount of a composition of claim 1 to the mammal, the drug being efficacious at treating the disease.

78. A method of treating a disease in a mammal comprising the administration of an effective amount of a composition of claim 67 to the mammal, the drug being efficacious at treating the disease.

79. A method of preventing a disease in a mammal comprising the administration of an effective amount of a composition of claim 1 to the mammal, the drug being efficacious at preventing the disease.

80. A method of preventing a disease in a mammal comprising the administration of an effective amount of a composition of 72 claim 67 to the mammal, the drug being efficacious at preventing the disease.

81. A method of claim 77 wherein the disease is selected from inflammatory conditions, autoimmune, neurological disorders, cancer, and benign hyperproliferative diseases.

82. A method of claim 77 wherein the disease is arthritis.

83. A method of claim 77 wherein the disease is multiple sclerosis.

84. The method of of claim 77 wherein the disease is Alzheimer's disease.

85. The method of claim 77 wherein the disease is psoriasis.

86. The method of claim 77 wherein the disease is cancer.

87. The method of claim 77 wherein the disease is stenosis or restenosis.

88. The method of claim 77 wherein the disease is benign hyperplasia.

89. The method of claim 77 wherein the hyperplasia is induced by a foreign body.

90. The method of claim 77 wherein the disease is cardiovascular disease.

91. The method of claim 77 wherein the disease is Inflammatory Bowel Disease.

92. The method of claim 77 wherein the composition is administered by a route selected from intravenous, intraarticular, intracutaneous, interstitial, subcutaneous, intramuscular injection, insertion into the rectum, oral, and implant into the body.

93. The method of claim 92 wherein the composition is administered by intravenous delivery of an aqueous micelle solution.

94. The method of claim 92 wherein the composition is administered by implanting a solid composition in the body, where the solid composition delivers drug to the body.

95. The method of claim 92 wherein the composition delivers paclitaxel or an analogue or derivative thereof to the body of the mammal.

96. The method of claim 93 wherein the composition delivers paclitaxel or an analogue or derivative thereof to the body of the mammal.

97. A method for enhancing the rate of dissolution of a water-soluble composition, wherein the composition comprises a hydrophobic drug and a polymer, the method comprising adding to the composition an amino acid that has a water-solubility of greater than about 2.5 g per 100 g water at 25° C.

98. A method for enhancing the rate of dissolution of a water-soluble composition, wherein the composition comprises a hydrophobic drug and a polymer, the method comprising adding to the composition an oligopeptide that has a water-solubility of greater than about 2.5 g per 100 g water at 25° C.
